SEQUENCE LISTING (22 sequences)

SEQ ID NO: 1 - DNA, 1730 bp, Talaromyces leycettanus
atggcgtccctcttctctttcaagatgtacaaggctgctctcgtcctgtcttctctcctggccgctacgcaggctcagcaggccggcactctcacgacggagacccatccgtccctgacatggcagcaatgctcggccggtggcagctgcaccacccagaacggcaaggtcgtcatcgatgcgaactggcgttgggtgcacagcacgagcggaagcaacaactgctacaccggcaatacctgggacgctaccctatgccctgacgatgtgacctgcgccgccaactgtgcgctggacggtgccgactactcgggcacctacggagtgaccaccagcggcaactccctccgcctcaacttcgtcacccaggcgtcacagaagaacgtcggctcccgtctttacctgatggagaatgacacaacctaccagatcttcaagctgctgaaccaggagttcacctttgatgtcgatgtgtccaacctgccgtaagtgacttaccatgaacccctgacgctatcttcttgttggctcccagctgactggccaattcaagctgcggcttgaacggtgctctctacctggtggccatggacgccgatggtggcatggccaagtaccccaccaacaaggctggtgccaagtacggtaccgggtactgcgactcccagtgtccccgcgacctcaagttcatcaatggcgaggccaacgtcgagggctggcagccgtcgtccaacgatcccaactctggcattggcaaccacggatcctgctgcgcggagatggatatctgggaggccaacagcatctccaatgctgtcactccccacccgtgcgacactcccggccaggtgatgtgcaccggtaacaactgcggtggcacatacagcactactcgctatgcgggcacttgcgatcccgacggctgcgacttcaacccctaccgcatgggcaaccacagcttctacggccctaaacagatcgtcgataccagctcgaagttcaccgtcgtgacgcagttcctcacggatgacggcacctccaccggcaccctctctgaaatccgccgcttctatgtccagaacggccaggtgatcccgaactcggtgtcgaccatcagtggcgtgagcggcaactccatcaccaccgagttctgcactgcccagaagcaggccttcggcgacacggacgacttctcaaagcacggcggcctgtccggcatgagcgctgccctctctcagggtatggttctggtcatgagtctgtgggatgatgtgagtttgatggacaaacatgcgcgttgacaaagagtcaagcagctgactgagatgttacagcacgccgccaacatgctctggctcgacagcacctacccgaccaacgcgacctcctccacccccggtgccgcccgtggaacctgcgacatctcgtccggtgtccctgcggatgtcgaatccaacgaccccaacgcctacgtggtctactcgaacatcaaggttggtcccatcggctcgaccttcagcagcagcggctctggatcttcttcctctagctccaccactaccacgaccaccgcttccccaaccaccacgacctcctccgcatcgagcaccggcactggagtggcacagcactggggccagtgtggtggacagggctggaccggccccacaacctgcgtcagcccttatacttgccaggagctgaacccttactactaccagtgtctgtaa

SEQ ID NO: 2 - protein, 532 aa, Talaromyces leycettanus
MASLFSFKMYKAALVLSSLLAATQAQQAGTLTTETHPSLTWQQCSAGGSCTTQNGKVVIDANWRWVHSTSGSNNCYTGNTWDATLCPDDVTCAANCALDGADYSGTYGVTTSGNSLRLNFVTQASQKNVGSRLYLMENDTTYQIFKLLNQEFTFDVDVSNLPCGLNGALYLVAMDADGGMAKYPTNKAGAKYGTGYCDSQCPRDLKFINGEANVEGWQPSSNDPNSGIGNHGSCCAEMDIWEANSISNAVTPHPCDTPGQVMCTGNNCGGTYSTTRYAGTCDPDGCDFNPYRMGNHSFYGPKQIVDTSSKFTVVTQFLTDDGTSTGTLSEIRRFYVQNGQVIPNSVSTISGVSGNSITTEFCTAQKQAFGDTDDFSKHGGLSGMSAALSQGMVLVMSLWDDHAANMLWLDSTYPTNATSSTPGAARGTCDISSGVPADVESNDPNAYVVYSNIKVGPIGSTFSSSGSGSSSSSSTTTTTTASPTTTTSSASSTGTGVAQHWGQCGGQGWTGPTTCVSPYTCQELNPYYYQCL

SEQ ID NO: 3 - DNA, 1898 bp, Taloromyces leycettanus
atgcggtctctcctggctcttgcccctaccctgctcgcgcctgttgttcaggctcagcaaaccatgtggggtcaatgtaagttcttttcactgcttaccatgtataatctttgatatcaagcatcatatctgactcacgttttaggcggtggtcagggctggaccggacctaccatctgtgtagcaggcgcgacatgcagcacacagaacccttgtaagtcgggccttcatcaaaacttcaacatcaccacctcgatggagcaggagttgacctgatctttacccttagggtatgcgcagtgcaccccagcacctaccgcgccgacgaccttgcaaacaacaactacgacgagctcgaaatcgtccacgaccacgagctcgaagtcgtccacgaccacaggtggaagtggcggtggaactacgacctcaacgtcagccaccatcaccgcggctccatctggtaacccatactccggataccagctctatgtgaaccaggaatactcgtccgaggtgtacgcgtctgctattccttcccttaccggcactctggtcgcgaaggcaagcgccgcggcagaggtgccatctttcctgtggctgtaagtttttttgaccttgaatgaacgccctgtcctctacgagtggccgcaggagctaattgagatgccaatgaacagggacactgcctccaaggtgccactgatgggcacttacttgcaggatatccaggcgaagaacgctgctggcgccaaccccccatatgccggtcaattcgtggtttacgacttgccggatcgtgattgcgctgcattggccagcaatggagagtactccattgctaacaatggtgttgccaactacaaggcttacatcgactccatccgcgcgcttcttgttcaatactcgaacgtccatgtcatccttgtgatcggtgagctattgcagtctcgctttaaagcatttgactagatcaatgtcgctaatggtacctaccgcacagagcccgacagcttggccaaccttgtcaccaacctgaatgttcagaagtgtgctaatgctcagagtgcttacctggagtgcatcaactatgccctcactcagttgaacctcaagaacgttgctatgtacatcgatgctggtgcgtgaaccttccctagtcagcccaaaataactgaaataaagagacggagtgtactgattgtcatgcaggtcatgctggatggctcggctggcccgccaaccttagcccggccgctcaactctttgcttccgtataccagaatgcaagctccccagctgccgttcgcggcctggcaaccaacgtggccaactataatgcctggtcgatcgccacttgcccatcttacacccaaggcgaccccaactgcgacgagcagaaatacatcaacgctctggctccattgcttcagcaacagggatggtcatcagttcactttatcaccgataccggtaagtctgcctgtcctgccaaccatgcgttcaagagcgttgcaatcctaaccatgctggtatcttccaggccgtaacggtgtccagcctaccaagcagaatgcctggggtgactggtgcaacgttatcggaaccggcttcggtgtccgtcccaccaccaacactggcgatccattggaggatgctttcgtctgggtcaagcctggtggtgagagtgatggtacttccaactccacttcgcctcgctacgacgcccactgcggttacagtgatgctcttcagcctgctcctgaggctggtacctggttcgaggtaagcttctgcatactgagatcgagaatcctgaaagggttaacctgctaatgcttcggtgtttgatataggcttactttgagcaactccttaccaacgccaacccctctttctaa

SEQ ID NO: 4 - protein, 464 aa, Taloromyces leycettanus
MRSLLALAPTLLAPVVQAQQTMWGQCGGQGWTGPTICVAGATCSTQNPWYAQCTPAPTAPTTLQTTTTTSSKSSTTTSSKSSTTTGGSGGGTTTSTSATITAAPSGNPYSGYQLYVNQEYSSEVYASAIPSLTGTLVAKASAAAEVPSFLWLDTASKVPLMGTYLQDIQAKNAAGANPPYAGQFVVYDLPDRDCAALASNGEYSIANNGVANYKAYIDSIRALLVQYSNVHVILVIEPDSLANLVTNLNVQKCANAQSAYLECINYALTQLNLKNVAMYIDAGHAGWLGWPANLSPAAQLFASVYQNASSPAAVRGLATNVANYNAWSIATCPSYTQGDPNCDEQKYINALAPLLQQQGWSSVHFITDTGRNGVQPTKQNAWGDWCNVIGTGFGVRPTTNTGDPLEDAFVWVKPGGESDGTSNSTSPRYDAHCGYSDALQPAPEAGTWFEAYFEQLLTNANPSF

SEQ ID NO: 5 - DNA, 3060 bp, Aspergillus fumigatus
atgagattcggttggctcgaggtggccgctctgacggccgcttctgtagccaatgcccaggtttgtgatgctttcccgtcattgtttcggatatagttgacaatagtcatggaaataatcaggaattggctttctctccaccattctacccttcgccttgggctgatggccagggagagtgggcagatgcccatcgacgcgccgtcgagatcgtttctcagatgacactggcggagaaggttaaccttacaacgggtactgggtgggttgcgacttttttgttgacagtgagctttcttcactgaccatctacacagatgggaaatggaccgatgcgtcggtcaaaccggcagcgttcccaggtaagcttgcaattctgcaacaacgtgcaagtgtagttgctaaaacgcggtggtgcagacttggtatcaactggggtctttgtggccaggattcccctttgggtatccgtttctgtgagctatacccgcggagtctttcagtccttgtattatgtgctgatgattgtctctgtatagctgacctcaactccgccttccctgctggtactaatgtcgccgcgacatgggacaagacactcgcctaccttcgtggcaaggccatgggtgaggaattcaacgacaagggcgtggacattttgctggggcctgctgctggtcctctcggcaaatacccggacggcggcagaatctgggaaggcttctctcctgatccggttctcactggtgtacttttcgccgaaactatcaagggtatccaagacgcgggtgtgattgctactgccaagcattacattctgaatgaacaggagcatttccgacaggttggcgaggcccagggatatggttacaacatcacggagacgatcagctccaacgtggatgacaagaccatgcacgagttgtacctttggtgagtagttgacactgcaaatgaggaccttgattgatttgactgacctggaatgcaggccctttgcagatgctgtgcgcggtaagattttccgtagacttgacctcgcgacgaagaaatcgctgacgaaccatcgtagctggcgttggcgctgtcatgtgttcctacaatcaaatcaacaacagctacggttgtcaaaacagtcaaactctcaacaagctcctcaaggctgagctgggcttccaaggcttcgtcatgagtgactggagcgctcaccacagcggtgtcggcgctgccctcgctgggttggatatgtcgatgcctggagacatttccttcgacgacggactctccttctggggcacgaacctaactgtcagtgttcttaacggcaccgttccagcctggcgtgtcgatgacatggctgttcgtatcatgaccgcgtactacaaggttggtcgtgaccgtcttcgtattccccctaacttcagctcctggacccgggatgagtacggctgggagcattctgctgtctccgagggagcctggaccaaggtgaacgacttcgtcaatgtgcagcgcagtcactctcagatcatccgtgagattggtgccgctagtacagtgctcttgaagaacacgggtgctcttcctttgaccggcaaggaggttaaagtgggtgttctcggtgaagacgctggttccaacccgtggggtgctaacggctgccccgaccgcggctgtgataacggcactcttgctatggcctggggtagtggtactgccaacttcccttaccttgtcacccccgagcaggctatccagcgagaggtcatcagcaacggcggcaatgtctttgctgtgactgataacggggctctcagccagatggcagatgttgcatctcaatccaggtgagtgcgggctcttagaaaaagaacgttctctgaatgaagttttttaaccattgcgaacagcgtgtctttggtgtttgtcaacgccgactctggagagggtttcatcagtgtcgacggcaacgagggtgaccgcaaaaatctcactctgtggaagaacggcgaggccgtcattgacactgttgtcagccactgcaacaacacgattgtggttattcacagtgttgggcccgtcttgatcgaccggtggtatgataaccccaacgtcactgccatcatctgggccggcttgcccggtcaggagagtggcaactccctggtcgacgtgctctatggccgcgtcaaccccagcgccaagaccccgttcacctggggcaagactcgggagtcttacggggctcccttgctcaccgagcctaacaatggcaatggtgctccccaggatgatttcaacgagggcgtcttcattgactaccgtcactttgacaagcgcaatgagacccccatttatgagtttggccatggcttgagctacaccacctttggttactctcaccttcgggttcaggccctcaatagttcgagttcggcatatgtcccgactagcggagagaccaagcctgcgccaacctatggtgagatcggtagtgccgccgactacctgtatcccgagggtctcaaaagaattaccaagtttatttacccttggctcaactcgaccgacctcgaggattcttctgacgacccgaactacggctgggaggactcggagtacattcccgaaggcgctagggatgggtctcctcaacccctcctgaaggctggcggcgctcctggtggtaaccctaccctttatcaggatcttgttagggtgtcggccaccataaccaacactggtaacgtcgccggttatgaagtccctcaattggtgagtgacccgcatgttccttgcgttgcaatttggctaactcgcttctagtatgtttcactgggcggaccgaacgagcctcgggtcgttctgcgcaagttcgaccgaatcttcctggctcctggggagcaaaaggtttggaccacgactcttaaccgtcgtgatctcgccaattgggatgtggaggctcaggactgggtcatcacaaagtaccccaagaaagtgcacgtcggcagctcctcgcgtaagctgcctctgagagcgcctctgccccgtgtctactag

SEQ ID NO: 6 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 7 - DNA, 835 bp, Penicillium sp. emersonii
atgctgtcttcgacgactcgcaccctcgcctttacaggccttgcgggccttctgtccgctcccctggtcaaggcccatggctttgtccagggcattgtcatcggtgaccaattgtaagtccctctcttgcagttctgtcgattaactgctggactgcttgcttgactccctgctgactcccaacagctacagcgggtacatcgtcaactcgttcccctacgaatccaacccaccccccgtcatcggctgggccacgaccgccaccgacctgggcttcgtcgacggcacaggataccaaggcccggacatcatctgccaccggaatgcgacgcccgcgccgctgacagcccccgtggccgccggcggcaccgtcgagctgcagtggacgccgtggccggacagccaccacggacccgtcatcacctacctggcgccgtgcaacggcaactgctcgaccgtcgacaagacgacgctggagttcttcaagatcgaccagcagggcctgatcgacgacacgagcccgccgggcacctgggcgtcggacaacctcatcgccaacaacaatagctggaccgtcaccattcccaacagcgtcgcccccggcaactacgtcctgcgccacgagatcatcgccctgcactcggccaacaacaaggacggcgcccagaactacccccagtgcatcaacatcgaggtcacgggcggcggctccgacgcgcctgagggtactctgggcgaggatctctaccatgacaccgacccgggcattctggtcgacatttacgagcccattgcgacgtataccattccggggccgcctgagccgacgttctag

SEQ ID NO: 8 - protein, 253 aa, Penicillium sp. emersonii
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 9 - DNA, 1520 bp, Talaromyces leycettanus
atggtccatctttcttccctggccctggctttggccgccggctcgcagctgtatgtgatccatgccatgactcgagaagtgctcccaaaactgactccaagtctcaatcttagtgcccaagctgcaggtcttaacactgctgccaaagcgattggaaagctctatttcggtaccgcaaccgacaacccggagctgtccgacagcacatacatgcaggagacggataacaccgatgatttcggccaactcaccccagctaactccatgaaggttcgctgacatcttagttcccccccccttttgggaatctgcgcggagatatgctgagccttcaaaactagtgggatgccaccgagccctctcagaacaccttcaccttcaccaacggtgatcagatcgcaaaccttgctaagagcaacggtcagatgctgagatgccacaacctggtgtggtacaaccagttgcccagctggggtaagcaaccggttctgttaatatcatcagcgtgaccgcatcgatcgtattgcgcggagattggaaagatttgcaagctaatgtcactacagtcaccagcggatcttggaccaatgccacgcttcttgcggccatgaagaaccacatcaccaacgttgtgacccactacaagggacagtgctacgcttgggatgttgtcaacgaaggtacgtttcgattcggcttccctcggaccgtatctgcaggcaaaaaggtcaatcaattgacaatcgtgatccccagctctcaacgatgatggcacctaccgatccaatgtcttctatcagtacatcggcgaggcatacattcccattgcctttgcgaccgctgccgccgccgatccaaacgcgaagctctactacaacgactacaacattgagtaccccggcgccaaggccaccgccgcccagaacatcgtcaagatggtcaaggcttacggcgcgaaaatcgacggtgtcggtctgcaatctcacttcatcgttggcagcacccctagccagagctcccagcagagcaacatggctgctttcaccgcgctcggcgtcgaggtcgccatcaccgaactggatatccgcatgacgttgccttccaccagtgctctcttggcccagcaatccaccgattaccagagcactgtgtcggcttgcgtgaacactccgaagtgcattggtatcaccctctgggactggaccgacaagtactcctgggttcccaacaccttctccggccaaggtgacgcctgcccctgggattctaactaccagaagaagcctgcctactacggtatcttgactgcgctcggaggcagcgcttccacctccaccaccaccactctggtgacctccaccaggacttcgactacgaccagcacttcggccacctccacgtctactggcgttgctcagcactggggccagtgcggtggtatcggctggacagggccgactacctgcgctagcccctacacctgccaggaactgaatccctactactaccagtgcctgtaa

SEQ ID NO: 10 - protein, 405 aa, Talaromyces leycettanus
MVHLSSLALALAAGSQLAQAAGLNTAAKAIGKLYFGTATDNPELSDSTYMQETDNTDDFGQLTPANSMKWDATEPSQNTFTFTNGDQIANLAKSNGQMLRCHNLVWYNQLPSWVTSGSWTNATLLAAMKNHITNVVTHYKGQCYAWDVVNEALNDDGTYRSNVFYQYIGEAYIPIAFATAAAADPNAKLYYNDYNIEYPGAKATAAQNIVKMVKAYGAKIDGVGLQSHFIVGSTPSQSSQQSNMAAFTALGVEVAITELDIRMTLPSTSALLAQQSTDYQSTVSACVNTPKCIGITLWDWTDKYSWVPNTFSGQGDACPWDSNYQKKPAYYGILTALGGSASTSTTTTLVTSTRTSTTTSTSATSTSTGVAQHWGQCGGIGWTGPTTCASPYTCQELNPYYYQCL

SEQ ID NO: 11 - DNA, 1197 bp, Trichophaea saccata
atgcgtaccttctcgtctcttctcggtgttgcccttctcttgggtgcagctaatgcccaggtcgcggtttggggacagtgtggtggcattggttactctggctcgacaacctgcgctgcgggaacgacttgtgttaagctgaacgactactactcccaatgccaacccggcggtaccactttgacaaccaccaccaaacccgccaccactaccactaccaccacggcaacttctccctcatcttctcccggattaaatgccctggcacaaaagagcggccggtacttcggtagtgcaactgacaacccagagctctccgatgcggcatacattgccatcctgagcaacaaaaacgagtttgggatcatcacgcctggaaactcgatgaaatgggatgctactgaaccgtcccgcgggagtttctcgttcactggtggacagcaaattgttgattttgcgcagggcaatgggcaggctatcagaggccatactcttgtctggtactcccagttgccgtcctgggttactagcggaaacttcgataaagctacattgacatcgatcatgcaaaatcacattacaactcttgtcagccactggaagggccagctcgcctactgggatgttgtcaacgaagcattcaacgatgatggcactttccgtcaaaacgtgttctacacaaccattggagaggactacatccagctcgccttcgaagccgcccgtgccgccgacccgaccgcaaagctctgcatcaacgactacaacatcgagggcactggagccaagtcaacagccatgtacaatctcgtctcgaagctgaaatccgccggcgttcccatcgactgtattggtgttcagggacacctcatcgtcggtgaagttcccaccaccatccaagcaaaccttgcccagtttgcgtctttgggtgtggatgtcgcgatcacggagctagatatcagaatgacgctgccatctacgactgcattgctccagcagcaggctaaggattacgtctcggttgttacagcctgcatgaatgttcccaggtgtatcggtatcaccatctgggactacactgataaatactcttgggtgccacaaaccttcagcggccagggcgatgcttgcccatgggatgccaacctgcagaagaagccagcctactccgctattgcgtctgctcttgcggcttga

SEQ ID NO: 12 - protein, 398 aa, Trichophaea saccata
MRTFSSLLGVALLLGAANAQVAVWGQCGGIGYSGSTTCAAGTTCVKLNDYYSQCQPGGTTLTTTTKPATTTTTTTATSPSSSPGLNALAQKSGRYFGSATDNPELSDAAYIAILSNKNEFGIITPGNSMKWDATEPSRGSFSFTGGQQIVDFAQGNGQAIRGHTLVWYSQLPSWVTSGNFDKATLTSIMQNHITTLVSHWKGQLAYWDVVNEAFNDDGTFRQNVFYTTIGEDYIQLAFEAARAADPTAKLCINDYNIEGTGAKSTAMYNLVSKLKSAGVPIDCIGVQGHLIVGEVPTTIQANLAQFASLGVDVAITELDIRMTLPSTTALLQQQAKDYVSVVTACMNVPRCIGITIWDYTDKYSWVPQTFSGQGDACPWDANLQKKPAYSAIASALAA

SEQ ID NO: 13 - DNA, 2391 bp, Taloromyces emersonii
atgatgactcccacggcgattctcaccgcagtggcggcgctcctgcccaccgcgacatgggcacaggataaccaaacctatgccaattactcgtcgcagtctcagccggacctgtttccccggaccgtcgcgaccatcgacctgtccttccccgactgtgagaatggcccgctcagcacgaacctggtgtgcaacaaatcggccgatccctgggcccgagctgaggccctcatctcgctctttaccctcgaagagctgattaacaacacccagaacaccgctcctggcgtgccccgtttgggtctgccccagtatcaggtgtggaatgaagctctgcacggactggaccgcgccaatttctcccattcgggcgaatacagctgggccacgtccttccccatgcccatcctgtcgatggcgtccttcaaccggaccctcatcaaccagattgcctccatcattgcaacgcaagcccgtgccttcaacaacgccggccgttacggccttgacagctatgcgcccaacatcaatggcttccgcagtcccctctggggccgtggacaggagacgcctggtgaggatgcgttcttcttgagttccacctatgcgtacgagtacatcacaggcctgcagggcggtgtcgacccagagcatgtcaagatcgtcgcgacggcgaagcacttcgccggctatgatctggagaactggggcaacgtctctcggctggggttcaatgctatcatcacgcagcaggatctctccgagtactacacccctcagttcctggcgtctgctcgatacgccaagacgcgcagcatcatgtgctcctacaatgcagtgaatggagtcccaagctgtgccaactccttcttcctccagacgcttctccgagaaaactttgacttcgttgacgacgggtacgtctcgtcggattgcgacgccgtctacaacgtcttcaacccacacggttacgcccttaaccagtcgggagccgctgcggactcgctcctagcaggtaccgatatcgactgtggtcagaccttgccgtggcacctgaatgagtccttcgtagaaggatacgtctcccgcggtgatatcgagaaatccctcacccgtctctactcaaacctggtgcgtctcggctactttgacggcaacaacagcgagtaccgcaacctcaactggaacgacgtcgtgactacggacgcctggaacatctcgtacgaggccgcggtggaaggtatcaccctgctcaagaacgacggaacgctgccgctgtccaagaaggtccgcagcattgcgctcatcggtccttgggccaatgccacggtgcagatgcagggtaactactatggaacgccaccgtatctgatcagtccgctggaagccgccaaggccagtgggttcacggtcaactatgcattcggtaccaacatctcgaccgattctacccagtggttcgcggaagccatcgcggcggcgaagaagtcggacgtgatcatctacgccggtggtattgacaacacgatcgaggcagagggacaggaccgcacggatctcaagtggccggggaaccagctggatctgatcgagcagctcagccaggtgggcaagcccttggtcgtcctgcagatgggcggtggccaggtggattcgtcgtcactcaaggccaacaagaatgtcaacgctctggtgtggggtggctatcccggacagtcgggtggtgcggccctgtttgacatccttacgggcaagcgtgcgccggccggtcgtctggtgagcacgcagtacccggccgagtatgcgacgcagttcccggccaacgacatgaacctgcgtccgaacggcagcaacccgggacagacatacatctggtacacgggcacgcccgtgtatgagttcggccacggtctgttctacacggagttccaggagtcggctgcggcgggcacgaacaagacgtcgactttcgacattctggaccttttctccacccctcatccgggatacgagtacatcgagcaggttccgttcatcaacgtgactgtggacgtgaagaacgtcggccacacgccatcgccgtacacgggtctgttgttcgcgaacacgacagccgggcccaagccgtacccgaacaaatggctcgtcgggttcgactggctgccgacgatccagccgggcgagactgccaagttgacgatcccggtgccgttgggcgcgattgcgtgggcggacgagaacggcaacaaggtggtcttcccgggcaactacgaattggcactgaacaatgagcgatcggtagtggtgtcgttcacgctgacgggcgatgcggcgactctagagaaatggcctttgtgggagcaggcggttccgggggtgctgcagcaataa

SEQ ID NO: 14 - protein, 796 aa, Talaromyces emersonii
MMTPTAILTAVAALLPTATWAQDNQTYANYSSQSQPDLFPRTVATIDLSFPDCENGPLSTNLVCNKSADPWARAEALISLFTLEELINNTQNTAPGVPRLGLPQYQVWNEALHGLDRANFSHSGEYSWATSFPMPILSMASFNRTLINQIASIIATQARAFNNAGRYGLDSYAPNINGFRSPLWGRGQETPGEDAFFLSSTYAYEYITGLQGGVDPEHVKIVATAKHFAGYDLENWGNVSRLGFNAIITQQDLSEYYTPQFLASARYAKTRSIMCSYNAVNGVPSCANSFFLQTLLRENFDFVDDGYVSSDCDAVYNVFNPHGYALNQSGAAADSLLAGTDIDCGQTLPWHLNESFVEGYVSRGDIEKSLTRLYSNLVRLGYFDGNNSEYRNLNWNDVVTTDAWNISYEAAVEGITLLKNDGTLPLSKKVRSIALIGPWANATVQMQGNYYGTPPYLISPLEAAKASGFTVNYAFGTNISTDSTQWFAEAIAAAKKSDVIIYAGGIDNTIEAEGQDRTDLKWPGNQLDLIEQLSQVGKPLVVLQMGGGQVDSSSLKANKNVNALVWGGYPGQSGGAALFDILTGKRAPAGRLVSTQYPAEYATQFPANDMNLRPNGSNPGQTYIWYTGTPVYEFGHGLFYTEFQESAAAGTNKTSTFDILDLFSTPHPGYEYIEQVPFINVTVDVKNVGHTPSPYTGLLFANTTAGPKPYPNKWLVGFDWLPTIQPGETAKLTIPVPLGAIAWADENGNKVVFPGNYELALNNERSVVVSFTLTGDAATLEKWPLWEQAVPGVLQQ

SEQ ID NO: 15 - DNA, 1507 bp, Trichoderma reesei
atggcgccctcagttacactgccgttgaccacggccatcctggccattgcccggctcgtcgccgcccagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactcgagggcgaatgccttgacccctcactcttgcacggccacggcctgcgactctgccggttgcggcttcaacccctatggcagcggctacaaaaggtgagcctgatgccactactacccctttcctggcgctctcgcggttttccatgctgacatggttttccagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacatcctggccaacaaccccaacacgcacgtcgtcttctccaacatccgctggggagacattgggtctactacgaactcgactgcgcccccgcccccgcctgcgtccagcacgacgttttcgactacacggaggagctcgacgacttcgagcagcccgagctgcacgcagactcactgggggcagtgcggtggcattgggtacagcgggtgcaagacgtgcacgtcgggcactacgtgccagtatagcaacgactgttcgtatccccatgcctgacgggagtgattttgagatgctaaccgctaaaatacagactactcgcaatgcctttag

SEQ ID NO: 16 - protein, 459 aa, Trichoderma reseei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 17 - DNA, 1507 bp, Trichoderma reesei; identical to SEQ ID NO: 15

SEQ ID NO: 18 - protein, 418 aa, Trichoderma Reesei
MNKSVAPLLLAASILYGGAAAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 19 - DNA, 1599 bp, Aspergillus Fumigatus
atgctggcctccaccttctcctaccgcatgtacaagaccgcgctcatcctggccgcccttctgggctctggccaggctcagcaggtcggtacttcccaggcggaagtgcatccgtccatgacctggcagagctgcacggctggcggcagctgcaccaccaacaacggcaaggtggtcatcgacgcgaactggcgttgggtgcacaaagtcggcgactacaccaactgctacaccggcaacacctgggacacgactatctgccctgacgatgcgacctgcgcatccaactgcgcccttgagggtgccaactacgaatccacctatggtgtgaccgccagcggcaattccctccgcctcaacttcgtcaccaccagccagcagaagaacattggctcgcgtctgtacatgatgaaggacgactcgacctacgagatgtttaagctgctgaaccaggagttcaccttcgatgtcgatgtctccaacctcccctgcggtctcaacggtgctctgtactttgtcgccatggacgccgacggtggcatgtccaagtacccaaccaacaaggccggtgccaagtacggtactggatactgtgactcgcagtgccctcgcgacctcaagttcatcaacggtcaggccaacgtcgaagggtggcagccctcctccaacgatgccaatgcgggtaccggcaaccacgggtcctgctgcgcggagatggatatctgggaggccaacagcatctccacggccttcaccccccatccgtgcgacacgcccggccaggtgatgtgcaccggtgatgcctgcggtggcacctacagctccgaccgctacggcggcacctgcgaccccgacggatgtgatttcaactccttccgccagggcaacaagaccttctacggccctggcatgaccgtcgacaccaagagcaagtttaccgtcgtcacccagttcatcaccgacgacggcacctccagcggcaccctcaaggagatcaagcgcttctacgtgcagaacggcaaggtgatccccaactcggagtcgacctggaccggcgtcagcggcaactccatcaccaccgagtactgcaccgcccagaagagcctgttccaggaccagaacgtcttcgaaaagcacggcggcctcgagggcatgggtgctgccctcgcccagggtatggttctcgtcatgtccctgtgggatgatcactcggccaacatgctctggctcgacagcaactacccgaccactgcctcttccaccactcccggcgtcgcccgtggtacctgcgacatctcctccggcgtccctgcggatgtcgaggcgaaccaccccgacgcctacgtcgtctactccaacatcaaggtcggccccatcggctcgaccttcaacagcggtggctcgaaccccggtggcggaaccaccacgacaactaccacccagcctactaccaccacgaccacggctggaaaccctggcggcaccggagtcgcacagcactatggccagtgtggtggaatcggatggaccggacccacaacctgtgccagcccttatacctgccagaagctgaatgattattactctcagtgcctgtag

SEQ ID NO: 20 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 21 - DNA, 1713 bp, Aspergillus fumigatus
atgaagcaccttgcatcttccatcgcattgactctactgttgcctgccgtgcaggcccagcagaccgtatggggccaatgtatgttctggctgtcactggaataagactgtatcaactgctgatatgcttctaggtggcggccaaggctggtctggcccgacgagctgtgttgccggcgcagcctgtagcacactgaatccctgtatgttagatatcgtcctgagtggagacttatactgacttccttagactacgctcagtgtatcccgggagccaccgcgacgtccaccaccctcacgacgacgacggcggcgacgacgacatcccagaccaccaccaaacctaccacgactggtccaactacatccgcacccaccgtgaccgcatccggtaaccctttcagcggctaccagctgtatgccaacccctactactcctccgaggtccatactctggccatgccttctctgcccagctcgctgcagcccaaggctagtgctgttgctgaagtgccctcatttgtttggctgtaagtggccttatcccaatactgagaccaactctctgacagtcgtagcgacgttgccgccaaggtgcccactatgggaacctacctggccgacattcaggccaagaacaaggccggcgccaaccctcctatcgctggtatcttcgtggtctacgacttgccggaccgtgactgcgccgctctggccagtaatggcgagtactcaattgccaacaacggtgtggccaactacaaggcgtacattgacgccatccgtgctcagctggtgaagtactctgacgttcacaccatcctcgtcatcggtaggccgtacacctccgttgcgcgccgcctttctctgacatcttgcagaacccgacagcttggccaacctggtgaccaacctcaacgtcgccaaatgcgccaatgcgcagagcgcctacctggagtgtgtcgactatgctctgaagcagctcaacctgcccaacgtcgccatgtacctcgacgcaggtatgcctcacttcccgcattctgtatcccttccagacactaactcatcaggccatgcgggctggctcggatggcccgccaacttgggccccgccgcaacactcttcgccaaagtctacaccgacgcgggttcccccgcggctgttcgtggcctggccaccaacgtcgccaactacaacgcctggtcgctcagtacctgcccctcctacacccagggagaccccaactgcgacgagaagaagtacatcaacgccatggcgcctcttctcaaggaagccggcttcgatgcccacttcatcatggatacctgtaagtgcttattccaatcgccgatgtgtgccgactaatcaatgtttcagcccggaatggcgtccagcccacgaagcaaaacgcctggggtgactggtgcaacgtcatcggcaccggcttcggtgttcgcccctcgactaacaccggcgatccgctccaggatgcctttgtgtggatcaagcccggtggagagagtgatggcacgtccaactcgacttccccccggtatgacgcgcactgcggatatagtgatgctctgcagcctgctcctgaggctggtacttggttccaggtatgtcatccattagccagatgagggataagtgactgacggacctaggcctactttgagcagcttctgaccaacgctaacccgtccttttaa

SEQ ID NO: 22 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

(19) United States
(12) Patent Application Publication
(10) Pub. No.: US 2017/0327855 A1
(43) Pub. Date: Nov. 16, 2017

(54) Milling Process

(71) Applicant: Novozymes A/S, Bagsvaerd (DK)

(72) Inventors: Zhen Long, Beijing (CN); James Lavigne, JR., Wake Forest, NC (US); Bernardo Vidal, JR., Wake Forest, NC (US); Brian R. Scott, West Sacramento, CA (US); Randall Scott Deinhammer, Wake Forest, NC (US); Thomas Patrick Gibbons, Wake Forest, NC (US); Chee-Leong Soong, Raleigh, NC (US); Yi Cao, Beijing (CN); Yu Zhang, Beijing (CN)

(73) Assignee: Novozymes A/S, Bagsvaerd (DK)

(21) Appl. No.: 15/527,515

(22) PCT Filed: Nov. 26, 2015
(86) PCT No.: PCT/CN2015/095622

§ 371 Date: May 17, 2017

(30) Foreign Application Priority Data
Nov. 26, 2014 (CN) PCT/CN2014/092267

(51) Int. Cl. C12P 19/04; C12P 19/14

(52) U.S. Cl. CPC C12P 19/04; C12Y 302/01091; C12Y 302/01037; C12P 19/14; C12Y 302/01008; C12Y 302/01021; C12P 2201/00

(57) ABSTRACT

The present invention provides process for treating crop kernels, comprising the steps of a) soaking kernels in water to produce soaked kernels; b) grinding the soaked kernels; c) treating the soaked kernels in the presence of an effective amount of an enzyme composition comprising: i) a protease, and ii) a cellulolytic composition, wherein step c) is performed before, during or after step b).

### REFERENCE TO SEQUENCE LISTING

This application contains a Sequence Listing in computer readable form. The computer readable form is incorporated herein by reference.

### FIELD OF THE INVENTION

The present invention relates to an improved process of treating crop kernels to provide a starch product of high quality suitable for conversion of starch into mono- and oligosaccharides, ethanol, sweeteners, etc. Further, the invention also relates to an enzyme composition comprising one or more enzyme activities suitable for the process of the invention and to the use of the composition of the invention.

### BACKGROUND OF THE INVENTION

Before starch, which is an important constituent in the kernels of most crops, such as corn, wheat, rice, sorghum bean, barley or fruit hulls, can be used for conversion of starch into saccharides, such as dextrose, fructose; alcohols, such as ethanol; and sweeteners, the starch must be made available and treated in a manner to provide a high purity starch. If starch contains more than 0.5% impurities, including the proteins, it is not suitable as starting material for starch conversion processes. To provide such pure and high quality starch product starting out from the kernels of crops, the kernels are often milled, as will be described further below.

Wet milling is often used for separating corn kernels into its four basic components: starch, germ, fiber and protein.

Typically wet milling processes comprise four basic steps. First the kernels are soaked or steeped for about 30 minutes to about 48 hours to begin breaking the starch and protein bonds. The next step in the process involves a coarse grind to break the pericarp and separate the germ from the rest of the kernel. The remaining slurry consisting of fiber, starch and protein is finely ground and screened to separate the fiber from the starch and protein. The starch is separated from the remaining slurry in hydrocyclones. The starch then can be converted to syrup or alcohol, or dried and sold as corn starch or chemically or physically modified to produce modified corn starch.

The use of enzymes has been suggested for the steeping step of wet milling processes. The commercial enzyme product Steepzyme® (available from Novozymes A/S) has been shown suitable for the first step in wet milling processes, i.e., the steeping step where corn kernels are soaked in water.

More recently, “enzymatic milling”, a modified wet-milling process that uses proteases to significantly reduce the total processing time during corn wet milling and eliminates the need for sulfur dioxide as a processing agent, has been developed. Johnston et al., Cereal Chem, 81, p. 626-632 (2004).

U.S. Pat. No. 6,566,125 discloses a method for obtaining starch from maize involving soaking maize kernels in water to produce soaked maize kernels, grinding the soaked maize kernels to produce a ground maize slurry, and incubating the ground maize slurry with enzyme (e.g., protease).

U.S. Pat. No. 5,066,218 discloses a method of milling grain, especially corn, comprising cleaning the grain, steeping the grain in water to soften it, and then milling the grain with a cellulase enzyme.

WO 2002/000731 discloses a process of treating crop kernels, comprising soaking the kernels in water for 1-12 hours, wet milling the soaked kernels and treating the kernels with one or more enzymes including an acidic protease.

WO 2002/000911 discloses a process of starch gluten separation, comprising subjecting mill starch to an acidic protease.

WO 2002/002644 discloses a process of washing a starch slurry obtained from the starch gluten separation step of a milling process, comprising washing the starch slurry with an aqueous solution comprising an effective amount of acidic protease.

WO 2014/082566 and WO 2014/082564 disclose cellulolytic compositions for use in wet milling.

There remains a need for improvement of processes for providing starch suitable for conversion into mono- and oligo-saccharides, ethanol, sweeteners, etc.

### SUMMARY OF THE INVENTION

The invention provides a process for treating crop kernels, comprising the steps of a) soaking kernels in water to produce soaked kernels; b) grinding the soaked kernels; c) treating the soaked kernels in the presence of an effective amount of an enzyme composition comprising: i) a protease, and ii) a cellulolytic composition, wherein step c) is performed before, during or after step b).

In one embodiment, the invention provides a process for treating crop kernels, comprising the steps of: a) soaking kernels in water to produce soaked kernels; b) grinding the soaked kernels; c) treating the soaked kernels in the presence of an effective amount of an enzyme composition comprising: i) a protease, ii) a cellulolytic composition, and wherein step c) is performed before, during or after step b).

In one embodiment, the invention provides a process for treating crop kernels, comprising the steps of: a) soaking kernels in water to produce soaked kernels; b) grinding the soaked kernels; c) treating the soaked kernels in the presence of an effective amount of an enzyme composition comprising: i) a protease, and ii) a cellulolytic composition, wherein step c) is performed before, during or after step b), and wherein the protease is present in a range of about 10% w/w to about 65% w/w of the total amount of enzyme protein.

In one embodiment, the invention provides the use of a cellulolytic composition to enhance the wet milling benefit of one or more enzymes.

### DETAILED DESCRIPTION OF THE INVENTION

Accordingly, it is an object of the invention to provide improved processes of treating crop kernels to provide starch of high quality.

In one embodiment, the enzyme compositions useful in the processes of the invention provide benefits including, improving starch yield and/or purity, improving gluten quality and/or yield, improving fiber, gluten, or steep water filtration, dewatering and evaporation, easier germ separation and/or better post-saccharification filtration, and process energy savings thereof.

Without wishing to be bound by theory, the present inventors have discovered that the role of proteases is more in separation of starch and protein from each other (protein from fiber, starch and protein interaction), e.g., by breaking the disulfide bonds. Use of protease leads to more pure starch and more pure gluten fractions, whereas use of cellulase and hemicellulase helps with separation of starch and protein complex from the fiber fraction, leading to much cleaner fiber and more starch plus gluten or mill starch yield. The combination of one of the above mentioned hemi-cellulase and/or cellulase with one of the above mentioned protease brings a particular combined benefit. In some embodiments, the enzyme blends useful in the process of the invention provide a synergistic effect.

Moreover, the present inventors have surprisingly found that the enzyme blends according to the invention provide the best reduction in fiber mass and the lowest protein content of the fiber due to better separation of both starch and protein fractions from the fiber fraction. Separating starch and gluten from fiber is valuable to the industry because fiber is the least valuable product of the wet milling process, and higher purity starch and protein is desirable.

Surprisingly, the present inventors have discovered that replacing some of the protease activity in an enzyme composition can provide an improvement over an otherwise similar composition containing predominantly protease activity alone. This can provide a benefit to the industry, e.g., on the basis of cost and ease of use.

### Definitions of Enzymes

Auxiliary Activity 9 polypeptide: The term “Auxiliary Activity 9 polypeptide” or “AA9 polypeptide” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic material by an enzyme having cellulolytic activity. Cellulolytic enhancing activity can be determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40° C.−80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST® 1.5 L (Novozymes A/S, Bagærd, Denmark) and beta-glucosidase as the source of the cellulolytic activity, wherein the beta-glucosidase is present at a weight of at least 2-5% protein of the cellulase protein loading. In one aspect, the beta-glucosidase is an Aspergillus oryzae beta-glucosidase (e.g., recombinantly produced in Aspergillus oryzae according to WO 02/095014). In another aspect, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASC.

AA9 polypeptide enhancing activity can also be determined according to WO 2013/028928 for high temperature compositions.

AA9 polypeptides enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

The AA9 polypeptide can also be used in the presence of a soluble activating divalent metal cation according to WO 2008/151043 or WO 2012/122518, e.g., manganese or copper. The AA9 polypeptide can be used in the presence of a dioxy compound, a bicylic compound, a heterocyclic compound, a nitrogen-containing compound, a quinone compound, a sulfurcontaining compound, or a liquor obtained from a pretreated cellulosic or hemicellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using pnitrophenyl-beta-D-glucopyranoside as substrate according to the procedure of Venturi et al., 2002, J. Basic Microbiol. 42: 55-66. One unit of beta-glucosidase is defined as 1.0 μmole of pnitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of pnitrophenolate anion produced per minute at 40° C., pH 5 from 1 mM p-nitrophenyl-beta-D-xyloside in 100 mM sodium citrate containing 0.01% TWEEN® 20.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teed, 1997, Trends in Biotechnology 15: 160-167; Teed et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C. 80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by AMINEX® HPX-87H column chromatography (Bio-Rad Laboratories, Inc., Hercules, Calif., USA).

Cellulosic material: The term “cellulosic material” means any material containing cellulose. Cellulose is a homopolymer of anyhdrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Endoglucanase: The term “endoglucanase” means a 4-(1,3; 1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can also be determined using carboxymethyl cellulose (CMC) as substrate according to the procedure of Ghose, 1987, Pure and Appl. Chem. 59: 257-268, at pH 5, 40° C.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GHA). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.−80° C., e.g., 40° C., 45° C., 50° C., 55° C., 60° C., 65° C., 70° C., 75° C., or 80° C., and a suitable pH such as 4-9, e.g., 4.0, 4.5, 5.0, 5.5, 6.0, 6.5, 7.0, 7.5, 8.0, 8.5, or 9.0.

Protease: The term “proteolytic enzyme” or “protease” means one or more (e.g., several) enzymes that break down the amide bond of a protein by hydrolysis of the peptide bonds that link amino acids together in a polypeptide chain. A protease may include, e.g., a metalloprotease, a trypsin-like serine protease, a subtilisin-like serine protease, and aspartic protease.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, 2006, Journal of the Science of Food and Agriculture 86(11): 1636-1647; Spanikova and Biely, 2006, FEBS Letters 580(19): 4597-4601; Herrmann et al., 1997, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. A common total xylanolytic activity assay is based on production of reducing sugars from polymeric 4-O-methyl glucuronoxylan as described in Bailey et al., 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Xylan degrading activity can be determined by measuring the increase in hydrolysis of birchwood xylan (Sigma Chemical Co., Inc., St. Louis, Mo., USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, Anal. Biochem. 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

### Other Definitions

Crop kernels: The term “crop kernels” includes kernels from, e.g., corn (maize), rice, barley, sorghum bean, fruit hulls, and wheat. Corn kernels are exemplary. A variety of corn kernels are known, including, e.g., dent corn, flint corn, pod corn, striped maize, sweet corn, waxy corn and the like.

In an embodiment, the corn kernel is yellow dent corn kernel. Yellow dent corn kernel has an outer covering referred to as the “Pericarp” that protects the germ in the kernels. It resists water and water vapour and is undesirable to insects and microorganisms.

The only area of the kernels not covered by the “Pericarp” is the “Tip Cap”, which is the attachment point of the kernel to the cob.

Germ: The “Germ” is the only living part of the corn kernel. It contains the essential genetic information, enzymes, vitamins, and minerals for the kernel to grow into a corn plant. In yellow dent corn, about 25 percent of the germ is corn oil. The endosperm covered surrounded by the germ comprises about 82 percent of the kernel dry weight and is the source of energy (starch) and protein for the germinating seed. There are two types of endosperm, soft and hard. In the hard endosperm, starch is packed tightly together. In the soft endosperm, the starch is loose.

Starch: The term “starch” means any material comprised of complex polysaccharides of plants, composed of glucose units that occurs widely in plant tissues in the form of storage granules, consisting of amylose and amylopectin, and represented as (C6H10O5)n, where n is any number.

Milled: The term “milled” refers to plant material which has been broken down into smaller particles, e.g., by crushing, fractionating, grinding, pulverizing, etc.

Grind or grinding: The term “grinding” means any process that breaks the pericarp and opens the crop kernel.

Steep or steeping: The term “steeping” means soaking the crop kernel with water and optionally SO₂.

Dry solids: The term “dry solids” is the total solids of a slurry in percent on a dry weight basis.

Oligosaccharide: The term “oligosaccharide” is a compound having 2 to 10 monosaccharide units.

Wet milling benefit: The term “wet milling benefit” means one or more of improved starch yield and/or purity, improved gluten quality and/or yield, improved fiber, gluten, or steep water filtration, dewatering and evaporation, easier germ separation and/or better post-saccharification filtration, and process energy savings thereof.

Allelic variant: The term “allelic variant” means any of two or more (e.g., several) alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Fragment: The term “fragment” means a polypeptide having one or more (e.g., several) amino acids absent from the amino and/or carboxyl terminus of a mature polypeptide, wherein the fragment has enzyme activity. In one aspect, a fragment contains at least 85%, e.g., at least 90% or at least 95% of the amino acid residues of the mature polypeptide of an enzyme.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide of a cellobiohydrolase I is amino acids 26 to 532 of SEQ ID NO: 2 based on the SignalP 3.0 program (Bendtsen et al., 2004, J. Mol. Biol. 340: 783-795) that predicts amino acids 1 to 25 of SEQ ID NO: 2 are a signal peptide. In another aspect, the mature polypeptide of a cellobiohydrolase II is amino acids 19 to 464 of SEQ ID NO: 4 based on the SignalP 3.0 program that predicts amino acids 1 to 18 of SEQ ID NO: 4 are a signal peptide. In another aspect, the mature polypeptide of a beta-glucosidase is amino acids 20 to 863 of SEQ ID NO: 6 based on the SignalP 3.0 program that predicts amino acids 1 to 19 of SEQ ID NO: 6 are a signal peptide. In another aspect, the mature polypeptide of a beta-glucosidase variant is amino acids 20 to 863 of SEQ ID NO: 36 based on the SignalP 3.0 program that predicts amino acids 1 to 19 of SEQ ID NO: 36 are a signal peptide. In another aspect, the mature polypeptide of an AA9 polypeptide is amino acids 26 to 253 of SEQ ID NO: 8 based on the SignalP 3.0 program that predicts amino acids 1 to 25 of SEQ ID NO: 8 are a signal peptide. In another aspect, the mature polypeptide of a GH10 xylanase is amino acids 21 to 405 of SEQ ID NO: 10 based on the SignalP 3.0 program that predicts amino acids 1 to 20 of SEQ ID NO: 10 are a signal peptide. In another aspect, the mature polypeptide of a GH10 xylanase is amino acids 20 to 398 of SEQ ID NO: 12 based on the SignalP 3.0 program that predicts amino acids 1 to 19 of SEQ ID NO: 12 are a signal peptide. In another aspect, the mature polypeptide of a beta-xylosidase is amino acids 22 to 796 of SEQ ID NO: 14 based on the SignalP 3.0 program that predicts amino acids 1 to 21 of SEQ ID NO: 14 are a signal peptide. In another aspect, the mature polypeptide of an endoglucanase I is amino acids 23 to 459 of SEQ ID NO: 16 based on the SignalP 3.0 program that predicts amino acids 1 to 22 of SEQ ID NO: 16 are a signal peptide. In another aspect, the mature polypeptide of an endoglucanase II is amino acids 22 to 418 of SEQ ID NO: 18 based on the SignalP 3.0 program that predicts amino acids 1 to 21 of SEQ ID NO: 18 are a signal peptide. In one aspect, the mature polypeptide of an A. fumigatus cellobiohydrolase I is amino acids 27 to 532 of SEQ ID NO: 20 based on the SignalP 3.0 program (Bendtsen et al., 2004, J. Mol. Biol. 340: 783-795) that predicts amino acids 1 to 26 of SEQ ID NO: 20 are a signal peptide. In another aspect, the mature polypeptide of an A. fumigatus cellobiohydrolase II is amino acids 20 to 454 of SEQ ID NO: 22 based on the SignalP 3.0 program that predicts amino acids 1 to 19 of SEQ ID NO: 22 are a signal peptide.

It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having enzyme activity. In one aspect, the mature polypeptide coding sequence of a cellobiohydrolase I is nucleotides 76 to 1727 of SEQ ID NO: 1 or the cDNA sequence thereof based on the SignalP 3.0 program (Bendtsen et al., 2004, supra) that predicts nucleotides 1 to 75 of SEQ ID NO: 1 encode a signal peptide. In another aspect, the mature polypeptide coding sequence of a cellobiohydrolase II is nucleotides 55 to 1895 of SEQ ID NO: 3 or the cDNA sequence thereof based on the SignalP 3.0 program that predicts nucleotides 1 to 54 of SEQ ID NO: 3 encode a signal peptide. In another aspect, the mature polypeptide coding sequence of a beta-glucosidase is nucleotides 58 to 3057 of SEQ ID NO: 5 or the cDNA sequence thereof based on the SignalP 3.0 program that predicts nucleotides 1 to 57 of SEQ ID NO: 5 encode a signal peptide. In another aspect, the mature polypeptide coding sequence of a beta-glucosidase variant is nucleotides 58 to 3057 of SEQ ID NO: 35 or the cDNA sequence thereof based on the SignalP 3.0 program that predicts nucleotides 1 to 57 of SEQ ID NO: 35 encode a signal peptide. In another aspect, the mature polypeptide coding sequence of an AA9 polypeptide is nucleotides 76 to 832 of SEQ ID NO: 7 or the cDNA sequence thereof based on the SignalP 3.0 program that predicts nucleotides 1 to 75 of SEQ ID NO: 7 encode a signal peptide. In another aspect, the mature polypeptide coding sequence of a GH10 xylanase is nucleotides 124 to 1517 of SEQ ID NO: 9 or the cDNA sequence thereof based on the SignalP 3.0 program that predicts nucleotides 1 to 123 of SEQ ID NO: 9 encode a signal peptide. In another aspect, the mature polypeptide coding sequence of a GH10 xylanase is nucleotides 58 to 1194 of SEQ ID NO: 11 based on the SignalP 3.0 program that predicts nucleotides 1 to 57 of SEQ ID NO: 11 encode a signal peptide. In another aspect, the mature polypeptide coding sequence of a beta-xylosidase is nucleotides 64 to 2388 of SEQ ID NO: 13 based on the SignalP 3.0 program that predicts nucleotides 1 to 63 of SEQ ID NO: 13 encode a signal peptide. In another aspect, the mature polypeptide coding sequence of an endoglucanase I is nucleotides 67 to 1504 of SEQ ID NO: 15 or the cDNA sequence thereof based on the SignalP 3.0 program that predicts nucleotides 1 to 66 of SEQ ID NO: 15 encode a signal peptide. In another aspect, the mature polypeptide coding sequence of an endoglucanase II is nucleotides 64 to 1504 of SEQ ID NO: 17 based on the SignalP 3.0 program that predicts nucleotides 1 to 63 of SEQ ID NO: 17 encode a signal peptide. In one aspect, the mature polypeptide coding sequence of an A. fumigatus cellobiohydrolase I is nucleotides 79 to 1596 of SEQ ID NO: 19 based on the SignalP 3.0 program (Bendtsen et al., 2004, supra) that predicts nucleotides 1 to 78 of SEQ ID NO: 19 encode a signal peptide. In another aspect, the mature polypeptide coding sequence of an A. fumigatus cellobiohydrolase II is nucleotides 58 to 1700 of SEQ ID NO: 21 or the cDNA sequence thereof based on the SignalP 3.0 program that predicts nucleotides 1 to 57 of SEQ ID NO: 21 encode a signal peptide.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence, wherein the subsequence encodes a fragment having enzyme activity. In one aspect, a subsequence contains at least 85%, e.g., at least 90% or at least 95% of the nucleotides of the mature polypeptide coding sequence of an enzyme.

Variant: The term “variant” means a polypeptide having enzyme activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

In one aspect, the variant differs by up to 10 amino acids, e.g., 1, 2, 3, 4, 5, 6, 7, 8, 9, or 10, from the mature polypeptide of a SEQ ID NO: as identified herein. In another embodiment, the present invention relates to variants of the mature polypeptide of a SEQ ID NO: herein comprising a substitution, deletion, and/or insertion at one or more (e.g., several) positions. In an embodiment, the number of amino acid substitutions, deletions and/or insertions introduced into the mature polypeptide of a SEQ ID NO: herein is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

### The Milling Process

The kernels are milled in order to open up the structure and to allow further processing and to separate the kernels into the four main constituents: starch, germ, fiber and protein.

In one embodiment, a wet milling process is used. Wet milling gives a very good separation of germ and meal (starch granules and protein) and is often applied at locations where there is a parallel production of syrups.

The inventors of the present invention have surprisingly found that the quality of the starch final product may be improved by treating crop kernels in the processes as described herein.

The processes of the invention result in comparison to traditional processes in a higher starch quality, in that the final starch product is more pure and/or a higher yield is obtained and/or less process time is used. Another advantage may be that the amount of chemicals, such as SO2 and NaHSO3, which need to be used, may be reduced or even fully removed.

### Wet Milling

Starch is formed within plant cells as tiny granules insoluble in water. When put in cold water, the starch granules may absorb a small amount of the liquid and swell. At temperatures up to about 50° C. to 75° C. the swelling may be reversible. However, with higher temperatures an irreversible swelling called “gelatinization” begins. Granular starch to be processed according to the present invention may be a crude starch-containing material comprising (e.g., milled) whole grains including non-starch fractions such as germ residues and fibers. The raw material, such as whole grains, may be reduced in particle size, e.g., by wet milling, in order to open up the structure and allowing for further processing. Wet milling gives a good separation of germ and meal (starch granules and protein) and is often applied at locations where the starch hydrolyzate is used in the production of, e.g., syrups.

In an embodiment the particle size is reduced to between 0.05-3.0 mm, preferably 0.1-0.5 mm, or so that at least 30%, preferably at least 50%, more preferably at least 70%, even more preferably at least 90% of the starch-containing material fits through a sieve with a 0.05-3.0 mm screen, preferably 0.1-0.5 mm screen.

More particularly, degradation of the kernels of corn and other crop kernels into starch suitable for conversion of starch into mono- and oligo-saccharides, ethanol, sweeteners, etc. consists essentially of four steps:

1. Steeping and germ separation,
2. Fiber washing and drying,
3. Starch gluten separation, and
4. Starch washing.

### 1. Steeping and Germ Separation

Corn kernels are softened by soaking in water for between about 30 minutes to about 48 hours, preferably 30 minutes to about 15 hours, such as about 1 hour to about 6 hours at a temperature of about 50° C., such as between about 45° C. to 60° C. During steeping, the kernels absorb water, increasing their moisture levels from 15 percent to 45 percent and more than doubling in size. The optional addition of e.g. 0.1 percent sulfur dioxide (SO2) and/or NaHSO3 to the water prevents excessive bacteria growth in the warm environment. As the corn swells and softens, the mild acidity of the steepwater begins to loosen the gluten bonds within the corn and release the starch. After the corn kernels are steeped they are cracked open to release the germ. The germ contains the valuable corn oil. The germ is separated from the heavier density mixture of starch, hulls and fiber essentially by “floating” the germ segment free of the other substances under closely controlled conditions. This method serves to eliminate any adverse effect of traces of corn oil in later processing steps.

In an embodiment of the invention the kernels are soaked in water for 2-10 hours, preferably about 3-5 hours at a temperature in the range between 40 and 60° C., preferably around 50° C.

In one embodiment, 0.01-1%, preferably 0.05-0.3%, especially 0.1% SO2 and/or NaHSO3 may be added during soaking.

### 2. Fiber Washing and Drying

To get maximum starch recovery, while keeping any fiber in the final product to an absolute minimum, it is necessary to wash the free starch from the fiber during processing. The fiber is collected, slurried and screened to reclaim any residual starch or protein.

### 3. Starch Gluten Separation

The starch-gluten suspension from the fiber-washing step, called mill starch, is separated into starch and gluten. Gluten has a low density compared to starch. By passing mill starch through a centrifuge, the gluten is readily spun out.

### 4. Starch Washing.

The starch slurry from the starch separation step contains some insoluble protein and much of solubles. They have to be removed before a top quality starch (high purity starch) can be made. The starch, with just one or two percent protein remaining, is diluted, washed 8 to 14 times, rediluted and washed again in hydroclones to remove the last trace of protein and produce high quality starch, typically more than 99.5% pure.

### Products

Wet milling can be used to produce, without limitation, corn steep liquor, corn gluten feed, germ, corn oil, corn gluten meal, cornstarch, modified corn starch, syrups such as corn syrup, and corn ethanol.

### Enzymes

The enzyme(s) and polypeptides described below are to be used in an “effective amount” in processes of the present invention. Below should be read in context of the enzyme disclosure in the “Definitions”-section above.

The enzyme composition of the present invention may be in any form suitable for use, such as, for example, a crude fermentation broth with or without cells removed, a cell lysate with or without cellular debris, a semi-purified or purified enzyme composition, or a host cell, e.g., Trichoderma host cell, as a source of the enzymes.

The enzyme composition may be a dry powder or granulate, a non-dusting granulate, a liquid, a stabilized liquid, or a stabilized protected enzyme. Liquid enzyme compositions may, for instance, be stabilized by adding stabilizers such as a sugar, a sugar alcohol or another polyol, and/or lactic acid or another organic acid according to established processes.

### Proteases

The protease may be any protease. Suitable proteases include microbial proteases, such as fungal and bacterial proteases. Preferred proteases are acidic proteases, i.e., proteases characterized by the ability to hydrolyze proteins under acidic conditions below pH 7. Preferred proteases are acidic endoproteases. An acid fungal protease is preferred, but also other proteases can be used.

The acid fungal protease may be derived from Aspergillus, Candida, Coriolus, Endothia, Enthomophtra, Irpex, Mucor, Penicillium, Rhizopus, Sclerotium, and Torulopsis. In particular, the protease may be derived from Aspergillus aculeatus (WO 95/02044), Aspergillus awamori (Hayashida et al., 1977, Agric. Biol. Chem. 42(5), 927-933), Aspergillus niger (see, e.g., Koaze et al., 1964, Agr. Biol. Chem. Japan 28: 216), Aspergillus saitoi (see, e.g., Yoshida, 1954, J. Agr. Chem. Soc. Japan 28: 66), or Aspergillus oryzae, such as the pepA protease; and acidic proteases from Mucor miehei or Mucor pusillus.

In an embodiment the acidic protease is a protease complex from A. oryzae sold under the tradename Flavourzyme® (from Novozymes A/S) or an aspartic protease from Rhizomucor miehei or Spezyme® FAN or GC 106 from Genencor Int.

In a preferred embodiment the acidic protease is an aspartic protease, such as an aspartic protease derived from a strain of Aspergillus, in particular A. aculeatus, especially A. aculeatus CBD 101.43.

Preferred acidic proteases are aspartic proteases, which retain activity in the presence of an inhibitor selected from the group consisting of pepstatin, Pefabloc, PMSF, or EDTA. Protease I derived from A. aculeatus CBS 101.43 is such an acidic protease.

In a preferred embodiment the process of the invention is carried out in the presence of the acidic Protease I derived from A. aculeatus CBS 101.43 in an effective amount.

In another embodiment the protease is derived from a strain of the genus Aspergillus, such as a strain of Aspergillus aculaetus, such as Aspergillus aculeatus CBS 101.43, such as the one disclosed in WO 95/02044, or a protease having at least 80%, such as at least 85%, such as at least 90%, preferably 95%, such as at least 96%, such as 97%, such as at least 98%, such as at least 99% identity to protease of WO 95/02044. In one aspect, the protease differs by up to 10 amino acids, e.g., 1, 2, 3, 4, 5, 6, 7, 8, 9, or 10, from the mature polypeptide of WO 95/02044. In another embodiment, the present invention relates to variants of the mature polypeptide of WO 95/02044 comprising a substitution, deletion, and/or insertion at one or more (e.g., several) positions. In an embodiment, the number of amino acid substitutions, deletions and/or insertions introduced into the mature polypeptide of WO 95/02044 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

The protease may be a neutral or alkaline protease, such as a protease derived from a strain of Bacillus. A particular protease is derived from Bacillus amyloliquefaciens and has the sequence obtainable at Swissprot as Accession No. P06832. The proteases may have at least 90% sequence identity to the amino acid sequence disclosed in the Swissprot Database, Accession No. P06832 such as at least 92%, at least 95%, at least 96%, at least 97%, at least 98%, or particularly at least 99% identity.

The protease may have at least 90% sequence identity to the amino acid sequence disclosed as sequence 1 in WO 2003/048353 such as at 92%, at least 95%, at least 96%, at least 97%, at least 98%, or particularly at least 99% identity.

The protease may be a papain-like protease selected from the group consisting of proteases within EC 3.4.22.* (cysteine protease), such as EC 3.4.22.2 (papain), EC 3.4.22.6 (chymopapain), EC 3.4.22.7 (asclepain), EC 3.4.22.14 (actinidain), EC 3.4.22.15 (cathepsin L), EC 3.4.22.25 (glycyl endopeptidase) and EC 3.4.22.30 (caricain).

In an embodiment, the protease is a protease preparation derived from a strain of Aspergillus, such as Aspergillus oryzae. In another embodiment the protease is derived from a strain of Rhizomucor, preferably Rhizomucor miehei. In another embodiment the protease is a protease preparation, preferably a mixture of a proteolytic preparation derived from a strain of Aspergillus, such as Aspergillus oryzae, and a protease derived from a strain of Rhizomucor, preferably Rhizomucor miehei.

Aspartic acid proteases are described in, for example, Handbook of Proteolytic Enzymes, Edited by A. J. Barrett, N. D. Rawlings and J. F. Woessner, Academic Press, San Diego, 1998, Chapter 270. Examples of aspartic acid proteases include, e.g., those disclosed in Berka et al., 1990, Gene 96: 313; Berka et al., 1993, Gene 125: 195-198; and Gomi et al., 1993, Biosci. Biotech. Biochem. 57: 1095-1100, which are hereby incorporated by reference.

The protease also may be a metalloprotease, which is defined as a protease selected from the group consisting of:

(a) proteases belonging to EC 3.4.24 (metalloendopeptidases); preferably EC 3.4.24.39 (acid metallo proteinases);
(b) metalloproteases belonging to the M group of the above Handbook;
(c) metalloproteases not yet assigned to clans (designation: Clan MX), or belonging to either one of clans MA, MB, MC, MD, ME, MF, MG, MH (as defined at pp. 989-991 of the above Handbook);
(d) other families of metalloproteases (as defined at pp. 1448-1452 of the above Handbook);
(e) metalloproteases with a HEXXH motif;
(f) metalloproteases with an HEFTH motif;
(g) metalloproteases belonging to either one of families M3, M26, M27, M32, M34, M35, M36, M41, M43, or M47 (as defined at pp. 1448-1452 of the above Handbook);
(h) metalloproteases belonging to the M28E family; and
(i) metalloproteases belonging to family M35 (as defined at pp. 1492-1495 of the above Handbook).

In other particular embodiments, metalloproteases are hydrolases in which the nucleophilic attack on a peptide bond is mediated by a water molecule, which is activated by a divalent metal cation. Examples of divalent cations are zinc, cobalt or manganese. The metal ion may be held in place by amino acid ligands. The number of ligands may be five, four, three, two, one or zero. In a particular embodiment the number is two or three, preferably three.

There are no limitations on the origin of the metalloprotease used in a process of the invention. In an embodiment the metalloprotease is classified as EC 3.4.24, preferably EC 3.4.24.39. In one embodiment, the metalloprotease is an acid-stable metalloprotease, e.g., a fungal acidstable metalloprotease, such as a metalloprotease derived from a strain of the genus Thermoascus, preferably a strain of Thermoascus aurantiacus, especially Thermoascus aurantiacus CGMCC No. 0670 (classified as EC 3.4.24.39). In another embodiment, the metalloprotease is derived from a strain of the genus Aspergillus, preferably a strain of Aspergillus oryzae.

In one embodiment the metalloprotease has a degree of sequence identity to amino acids 159 to 177, or preferably amino acids 1 to 177 (the mature polypeptide) of SEQ ID NO: 1 of WO 2010/008841 (a Thermoascus aurantiacus metalloprotease) of at least 80%, at least 82%, at least 85%, at least 90%, at least 95%, at least 97%, at least 98%, or at least 99%; and which have metalloprotease activity.

The Thermoascus aurantiacus metalloprotease is a preferred example of a metalloprotease suitable for use in a process of the invention. Another metalloprotease is derived from Aspergillus oryzae and comprises SEQ ID NO: 11 disclosed in WO 2003/048353, or amino acids 23-353; 23-374; 23-397; 1-353; 1-374; 1-397; 177-353; 177-374; or 177-397 thereof, and SEQ ID NO: 10 disclosed in WO 2003/048353.

Another metalloprotease suitable for use in a process of the invention is the Aspergillus oryzae metalloprotease comprising SEQ ID NO: 5 of WO 2010/008841, or a metalloprotease is an isolated polypeptide which has a degree of identity to SEQ ID NO: SEQ ID NO: 5 of at least about 80%, at least 82%, at least 85%, at least 90%, at least 95%, at least 97%; at least 98%, or at least 99% and which have metalloprotease activity. In particular embodiments, the metalloprotease consists of the amino acid sequence of SEQ ID NO: 5 5.

In a particular embodiment, a metalloprotease has an amino acid sequence that differs by forty, thirty-five, thirty, twenty-five, twenty, or by fifteen amino acids from amino acids 159 to 177, or +1 to 177 of the amino acid sequences of the Thermoascus aurantiacus or Aspergillus oryzae metalloprotease.

In another embodiment, a metalloprotease has an amino acid sequence that differs by ten, or by nine, or by eight, or by seven, or by six, or by five amino acids from amino acids 159 to 177, or +1 to 177 of the amino acid sequences of these metalloproteases, e.g., by four, by three, by two, or by one amino acid.

In particular embodiments, the metalloprotease a) comprises or b) consists of

i) the amino acid sequence of amino acids 159 to 177, or +1 to 177 of SEQ ID NO: 1 of WO 2010/008841;
ii) the amino acid sequence of amino acids 23-353, 23-374, 23-397, 1-353, 1-374, 1-397, 177-353, 177-374, or 177-397 of SEQ ID NO: 3 of WO 2010/008841;
iii) the amino acid sequence of SEQ ID NO: 5 of WO 2010/008841; or
allelic variants, or fragments, of the sequences of i), ii), and iii) that have protease activity.

A fragment of amino acids 159 to 177, or +1 to 177 of SEQ ID NO: 1 of WO 2010/008841 or of amino acids 23-353, 23-374, 23-397, 1-353, 1-374, 1-397, 177-353, 177-374, or 177-397 of SEQ ID NO: 3 of WO 2010/008841; is a polypeptide having one or more amino acids deleted from the amino and/or carboxyl terminus of these amino acid sequences. In one embodiment a fragment contains at least 75 amino acid residues, or at least 100 amino acid residues, or at least 125 amino acid residues, or at least 150 amino acid residues, or at least 160 amino acid residues, or at least 165 amino acid residues, or at least 170 amino acid residues, or at least 175 amino acid residues.

In another embodiment, the metalloprotease is combined with another protease, such as a fungal protease, preferably an acid fungal protease.

In a preferred embodiment the protease is S53 protease 3 from Meripilus giganteus disclosed in Examples 1 and 2 in WO 2014/037438 (which is hereby incorporated by reference), e.g., a polypeptide having at least 90% sequence identity to the polypeptide of SEQ ID NO: 5, SEQ ID NO: 6 of WO 2014/037438, or the mature polypeptide of SEQ ID NO: 2 or SEQ ID NO: 4 of WO 2014/037438;

(b) a polypeptide encoded by a polynucleotide that hybridizes under high stringency conditions, or very high stringency conditions with
(i) the mature polypeptide coding sequence of SEQ ID NO: 1 of WO 2014/037438,
(ii) the mature polypeptide coding sequence of SEQ ID NO: 3 of WO 2014/037438,
(iii) the full-length complementary strand of (i) or (ii);
(c) a polypeptide encoded by a polynucleotide having at least 90% sequence identity to the mature polypeptide coding sequence of SEQ ID NO: 1 or SEQ ID NO: 3 of WO 2014/037438;
(d) a variant of the polypeptide of SEQ ID NO: 5, SEQ ID NO: 6 of WO 2014/037438, or the mature polypeptide of SEQ ID NO: 2 or SEQ ID NO: 4 of WO 2014/037438 comprising a substitution, deletion, and/or insertion at one or more (several) positions; and
(e) a fragment of a polypeptide of (a), (b), or (c) having protease activity.

Commercially available products include ALCALASE®, ESPERASE™, FLAVOURZYME™, NEUTRASE®, RENNILASE®, NOVOZYM™ FM 2.0 L, and iZyme BA (available from Novozymes A/S, Denmark) and GC106™ and SPEZYME™ FAN from Genencor International, Inc., USA.

The protease may be present in an amount of 0.0001-1 mg enzyme protein per g dry solids (DS) kernels, preferably 0.001 to 0.1 mg enzyme protein per g DS kernels.

In an embodiment, the protease is an acidic protease added in an amount of 1-20,000 HUT/100 g DS kernels, such as 1-10,000 HUT/100 g DS kernels, preferably 300-8,000 HUT/100 g DS kernels, especially 3,000-6,000 HUT/100 g DS kernels, or 4,000-20,000 HUT/100 g DS kernels acidic protease, preferably 5,000-10,000 HUT/100 g, especially from 6,000-16,500 HUT/100 g DS kernels.

### Cellulolytic Compositions

The present invention relates to use of cellulolytic compositions as described in e.g., United States Patent Application No. 61/909,114 filed Nov. 26, 2013 and U.S. Patent Application No. 62/009,018 filed Jun. 6, 2014.

In particular, according to an embodiment, the present invention relates to use of enzyme compositions, comprising: (A) (i) a cellobiohydrolase I, (ii) a cellobiohydrolase II, and (iii) at least one enzyme selected from the group consisting of a beta-glucosidase or a variant thereof, an AA9 polypeptide having cellulolytic enhancing activity, a GH10 xylanase, and a beta-xylosidase; (B) (i) a GH10 xylanase and (ii) a beta-xylosidase; or (C) (i) a cellobiohydrolase I, (ii) a cellobiohydrolase II, (iii) a GH10 xylanase, and (iv) a beta-xylosidase;

wherein the cellobiohydrolase I is selected from the group consisting of: (i) a cellobiohydrolase I comprising or consisting of the mature polypeptide of SEQ ID NO: 2; (ii) a cellobiohydrolase I comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 2; (iii) a cellobiohydrolase I encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 1; and (iv) a cellobiohydrolase I encoded by a polynucleotide that hybridizes under at least high stringency conditions, e.g., very high stringency conditions, with the mature polypeptide coding sequence of SEQ ID NO: 1 or the full-length complement thereof;

wherein the cellobiohydrolase II is selected from the group consisting of: (i) a cellobiohydrolase II comprising or consisting of the mature polypeptide of SEQ ID NO: 4; (ii) a cellobiohydrolase II comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 4; (iii) a cellobiohydrolase II encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 3; and (iv) a cellobiohydrolase II encoded by a polynucleotide that hybridizes under at least high stringency conditions, e.g., very high stringency conditions, with the mature polypeptide coding sequence of SEQ ID NO: 3 or the full-length complement thereof;

wherein the beta-glucosidase is selected from the group consisting of: (i) a betaglucosidase comprising or consisting of the mature polypeptide of SEQ ID NO: 6; (ii) a betaglucosidase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 6; (iii) a beta-glucosidase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 5; and (iv) a beta-glucosidase encoded by a polynucleotide that hybridizes under at least high stringency conditions, e.g., very high stringency conditions, with the mature polypeptide coding sequence of SEQ ID NO: 5 or the full-length complement thereof;

wherein the xylanase is selected from the group consisting of: (i) a xylanase comprising or consisting of the mature polypeptide of SEQ ID NO: 10 or the mature polypeptide of SEQ ID NO: 12; (ii) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 10 or the mature polypeptide of SEQ ID NO: 12; (iii) a xylanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 9 or the mature polypeptide coding sequence of SEQ ID NO: 11; and (iv) a xylanase encoded by a polynucleotide that hybridizes under at least high stringency conditions, e.g., very high stringency conditions, with the mature polypeptide coding sequence of SEQ ID NO: 9 or the mature polypeptide coding sequence of SEQ ID NO: 11; or the full-length complement thereof; and

wherein the beta-xylosidase is selected from the group consisting of: (i) a betaxylosidase comprising or consisting of the mature polypeptide of SEQ ID NO: 14; (ii) a betaxylosidase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 14; (iii) a beta-xylosidase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 13; and (iv) a beta-xylosidase encoded by a polynucleotide that hybridizes under at least high stringency conditions, e.g., very high stringency conditions, with the mature polypeptide coding sequence of SEQ ID NO: 13 or the full-length complement thereof.

In one aspect, the AA9 (GH61) polypeptide is any AA9 polypeptide having cellulolytic enhancing activity. Examples of AA9 polypeptides include, but are not limited to, AA9 polypeptides from Thielavia terrestris (WO 2005/074647, WO 2008/148131, and WO 2011/035027), Thermoascus aurantiacus (WO 2005/074656 and WO 2010/065830), Trichoderma reesei (WO 2007/089290 and WO 2012/149344), Myceliophthora thermophila (WO 2009/085935, WO 2009/085859, WO 2009/085864, WO 2009/085868, WO 2009/033071, WO 2012/027374, and WO 2012/068236), Aspergillus fumigatus (WO 2010/138754), Penicillium pinophilum (WO 2011/005867), Thermoascus sp. (WO 2011/039319), Penicillium sp. (emersonii) (WO 2011/041397 and WO 2012/000892), Thermoascus crustaceous (WO 2011/041504), Aspergillus aculeatus (WO 2012/125925), Thermomyces lanuginosus (WO 2012/113340, WO 2012/129699, WO 2012/130964, and WO 2012/129699), Aurantiporus alborubescens (WO 2012/122477), Trichophaea saccata (WO 2012/122477), Penicillium thomii (WO 2012/122477), Talaromyces stipitatus (WO 2012/135659), Humicola insolens (WO 2012/146171), Malbranchea cinnamomea (WO 2012/101206), Talaromyces leycettanus (WO 2012/101206), and Chaetomium thermophilum (WO 2012/101206), Talaromyces emersonii (WO 2012/000892), Trametes versicolor (WO 2012/092676 and WO 2012/093149), and Talaromyces thermophilus (WO 2012/129697 and WO 2012/130950); which are incorporated herein by reference in their entireties.

In another aspect, the AA9 polypeptide having cellulolytic enhancing activity is selected from the group consisting of: (i) an AA9 polypeptide having cellulolytic enhancing activity comprising or consisting of the mature polypeptide of SEQ ID NO: 8; (ii) an AA9 polypeptide having cellulolytic enhancing activity comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 8; (iii) an AA9 polypeptide having cellulolytic enhancing activity encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 7; and (iv) an AA9 polypeptide having cellulolytic enhancing activity encoded by a polynucleotide that hybridizes under at least high stringency conditions, e.g., very high stringency conditions, with the mature polypeptide coding sequence of SEQ ID NO: 7 or the full-length complement thereof.

In one embodiment, the enzyme composition comprises a cellobiohydrolase I, a cellobiohydrolase II, and a beta-glucosidase or a variant thereof.

In another embodiment, the enzyme composition comprises a cellobiohydrolase I, a cellobiohydrolase II, and an AA9 polypeptide having cellulolytic enhancing activity.

In another embodiment, the enzyme composition comprises a cellobiohydrolase I, a cellobiohydrolase II, and a GH10 xylanase.

In another embodiment, the enzyme composition comprises a cellobiohydrolase I, a cellobiohydrolase II, and a beta-xylosidase.

In another embodiment, the enzyme composition comprises a cellobiohydrolase I, a cellobiohydrolase II, a beta-glucosidase or a variant thereof, and an AA9 polypeptide having cellulolytic enhancing activity.

In another embodiment, the enzyme composition comprises a cellobiohydrolase I, a cellobiohydrolase II, a beta-glucosidase or a variant thereof, and a GH10 xylanase.

In another embodiment, the enzyme composition comprises a cellobiohydrolase I, a cellobiohydrolase II, a beta-glucosidase or a variant thereof, and a beta-xylosidase.

In another embodiment, the enzyme composition comprises a cellobiohydrolase I, a cellobiohydrolase II, an AA9 polypeptide having cellulolytic enhancing activity, and a GH10 xylanase.

In another embodiment, the enzyme composition comprises a cellobiohydrolase I, a cellobiohydrolase II, an AA9 polypeptide having cellulolytic enhancing activity, and a betaxylosidase.

In another embodiment, the enzyme composition comprises a cellobiohydrolase I, a cellobiohydrolase II, a GH10 xylanase, and a beta-xylosidase.

In another embodiment, the enzyme composition comprises a cellobiohydrolase I, a cellobiohydrolase II, a beta-glucosidase or a variant thereof, an AA9 polypeptide having cellulolytic enhancing activity, and a GH10 xylanase.

In another embodiment, the enzyme composition comprises a cellobiohydrolase I, a cellobiohydrolase II, a beta-glucosidase or a variant thereof, an AA9 polypeptide having cellulolytic enhancing activity, and a beta-xylosidase.

In another embodiment, the enzyme composition comprises a cellobiohydrolase I, a cellobiohydrolase II, a beta-glucosidase or a variant thereof, a GH10 xylanase, and a betaxylosidase.

In another embodiment, the enzyme composition comprises a cellobiohydrolase I, a cellobiohydrolase II, an AA9 polypeptide having cellulolytic enhancing activity, a GH10 xylanase, and a beta-xylosidase.

In another embodiment, the enzyme composition comprises a cellobiohydrolase I, a cellobiohydrolase II, a beta-glucosidase or a variant thereof, an AA9 polypeptide having cellulolytic enhancing activity, a GH10 xylanase, and a beta-xylosidase.

Each of the enzyme compositions described above may further or even further comprise an endoglucanase I, an endoglucanase II, or an endoglucanase I and an endoglucanase II.

In one aspect, the endoglucanase I is selected from the group consisting of: (i) an endoglucanase I comprising or consisting of the mature polypeptide of SEQ ID NO: 16; (ii) an endoglucanase I comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 16; (iii) an endoglucanase I encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 15; and (iv) an endoglucanase I encoded by a polynucleotide that hybridizes under at least high stringency conditions, e.g., very high stringency conditions, with the mature polypeptide coding sequence of SEQ ID NO: 15 or the full-length complement thereof.

In another aspect, the endoglucanase II is selected from the group consisting of: (i) an endoglucanase II comprising or consisting of the mature polypeptide of SEQ ID NO: 18; (ii) an endoglucanase II comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 18; (iii) an endoglucanase II encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 17; and (iv) an endoglucanase II encoded by a polynucleotide that hybridizes under at least high stringency conditions, e.g., very high stringency conditions, with the mature polypeptide coding sequence of SEQ ID NO: 17 or the full-length complement thereof.

In particular, according to an embodiment, the present invention relates to use of enzyme compositions, comprising: (A) (i) an Aspergillus fumigatus cellobiohydrolase I; (ii) an Aspergillus fumigatus cellobiohydrolase II; (iii) an Aspergillus fumigatus beta-glucosidase or variant thereof; (iv) a Penicillium sp. AA9 polypeptide having cellulolytic enhancing activity; (v) a Trichophaea saccata GH10 xylanase; and (vi) a Talaromyces emersonii beta-xylosidase; or homologs thereof; (B) (i) an Aspergillus fumigatus cellobiohydrolase I; (ii) an Aspergillus fumigatus cellobiohydrolase II; (iii) a Trichophaea saccata GH10 xylanase; and (iv) a Talaromyces emersonii beta-xylosidase; or homologs thereof; or (C) (i) a Trichophaea saccata GH10 xylanase; and (ii) a Talaromyces emersonii beta-xylosidase; or homologs thereof.

In one aspect, the Aspergillus fumigatus cellobiohydrolase I or a homolog thereof is selected from the group consisting of: (i) a cellobiohydrolase I comprising or consisting of the mature polypeptide of SEQ ID NO: 20; (ii) a cellobiohydrolase I comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 20; (iii) a cellobiohydrolase I encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 19; and (iv) a cellobiohydrolase I encoded by a polynucleotide that hybridizes under at least high stringency conditions, e.g., very high stringency conditions, with the mature polypeptide coding sequence of SEQ ID NO: 19 or the full-length complement thereof.

In another aspect, the Aspergillus fumigatus cellobiohydrolase II or a homolog thereof is selected from the group consisting of: (i) a cellobiohydrolase II comprising or consisting of the mature polypeptide of SEQ ID NO: 22; (ii) a cellobiohydrolase II comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 22; (iii) a cellobiohydrolase II encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 21; and (iv) a cellobiohydrolase II encoded by a polynucleotide that hybridizes under at least high stringency conditions, e.g., very high stringency conditions, with the mature polypeptide coding sequence of SEQ ID NO: 21 or the full-length complement thereof.

In another aspect, the Aspergillus fumigatus beta-glucosidase or a homolog thereof is selected from the group consisting of: (i) a beta-glucosidase comprising or consisting of the mature polypeptide of SEQ ID NO: 6; (ii) a beta-glucosidase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 6; (iii) a beta-glucosidase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 5; and (iv) a beta-glucosidase encoded by a polynucleotide that hybridizes under at least high stringency conditions, e.g., very high stringency conditions, with the mature polypeptide coding sequence of SEQ ID NO: 5 or the full-length complement thereof.

In another aspect, the Penicillium sp. (emersonh) AA9 polypeptide having cellulolytic enhancing activity or a homolog thereof is selected from the group consisting of: (i) an AA9 polypeptide having cellulolytic enhancing activity comprising or consisting of the mature polypeptide of SEQ ID NO: 8; (ii) an AA9 polypeptide having cellulolytic enhancing activity comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 8; (iii) an AA9 polypeptide having cellulolytic enhancing activity encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 7; and (iv) an AA9 polypeptide having cellulolytic enhancing activity encoded by a polynucleotide that hybridizes under at least high stringency conditions, e.g., very high stringency conditions, with the mature polypeptide coding sequence of SEQ ID NO: 7 or the full-length complement thereof.

In another aspect, the Trichophaea saccata xylanase or a homolog thereof is selected from the group consisting of: (i) a xylanase comprising or consisting of the mature polypeptide of SEQ ID NO: 12; (ii) a xylanase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 12; (iii) a xylanase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 11; and (iv) a xylanase encoded by a polynucleotide that hybridizes under at least high stringency conditions, e.g., very high stringency conditions, with the mature polypeptide coding sequence of SEQ ID NO: 11; or the full-length complement thereof.

In another aspect, the Talaromyces emersonii beta-xylosidase or a homolog thereof is selected from the group consisting of: (i) a beta-xylosidase comprising or consisting of the mature polypeptide of SEQ ID NO: 14; (ii) a beta-xylosidase comprising or consisting of an amino acid sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 14; (iii) a beta-xylosidase encoded by a polynucleotide comprising or consisting of a nucleotide sequence having at least 70%, e.g., at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 13; and (iv) a beta-xylosidase encoded by a polynucleotide that hybridizes under at least high stringency conditions, e.g., very high stringency conditions, with the mature polypeptide coding sequence of SEQ ID NO: 13 or the full-length complement thereof.

In another aspect, the enzyme composition further or even further comprises a Trichoderma endoglucanase I or a homolog thereof. In another aspect, the enzyme composition further comprises a Trichoderma reesei endoglucanase I or a homolog thereof. In another aspect, the enzyme composition further comprises a Trichoderma reesei Cel7B endoglucanase I (GENBANK™ accession no. M15665) or homolog thereof. In another aspect, the Trichoderma reesei endoglucanase I or a homolog thereof is native to the host cell.

In another aspect, the enzyme composition further or even further comprises a Trichoderma endoglucanase II or a homolog thereof. In another aspect, the enzyme composition further comprises a Trichoderma reesei endoglucanase II or a homolog thereof. In another aspect, the enzyme composition further comprises a Trichoderma reesei Cel5A endoglucanase II (GENBANK™ accession no. M19373) or a homolog thereof. In another aspect, the Trichoderma reesei endoglucanase II or a homolog thereof is native to the host cell.

A protein engineered variant of an enzyme above (or protein) may also be used.

In one aspect, the variant is a beta-glucosidase variant. In another aspect, the variant is an Aspergillus fumigatus beta-glucosidase variant. In another aspect, the A. fumigatus betaglucosidase variant comprises a substitution at one or more (several) positions corresponding to positions 100, 283, 456, and 512 of SEQ ID NO: 6, wherein the variant has beta-glucosidase activity.

In an embodiment, the variant has sequence identity of at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100%, to the amino acid sequence of the parent beta-glucosidase.

In another embodiment, the variant has at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100%, sequence identity to the mature polypeptide of SEQ ID NO: 6.

For purposes of the present invention, the mature polypeptide disclosed in SEQ ID NO: 6 is used to determine the corresponding amino acid residue in another beta-glucosidase. The amino acid sequence of another beta-glucosidase is aligned with the mature polypeptide disclosed in SEQ ID NO: 6, and based on the alignment, the amino acid position number corresponding to any amino acid residue in the mature polypeptide disclosed in SEQ ID NO: 6 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are a gap open penalty of 10, a gap extension penalty of 0.5, and the EBLOSUM62 (EMBOSS version of BLOSUM62) substitution matrix.

Identification of the corresponding amino acid residue in another beta-glucosidase can be determined by alignment of multiple polypeptide sequences using several computer programs including, but not limited to, MUSCLE (multiple sequence comparison by logexpectation; version 3.5 or later; Edgar, 2004, Nucleic Acids Research 32: 1792-2797), MAFTT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

In one aspect, a variant comprises a substitution at one or more (several) positions corresponding to positions 100, 283, 456, and 512. In another aspect, a variant comprises a substitution at two positions corresponding to any of positions 100, 283, 456, and 512. In another aspect, a variant comprises a substitution at three positions corresponding to any of positions 100, 283, 456, and 512. In another aspect, a variant comprises a substitution at each position corresponding to positions 100, 283, 456, and 512.

In another aspect, the variant comprises or consists of a substitution at a position corresponding to position 100. In another aspect, the amino acid at a position corresponding to position 100 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises or consists of the substitution F100D of the mature polypeptide of SEQ ID NO: 6.

In another aspect, the variant comprises or consists of a substitution at a position corresponding to position 283. In another aspect, the amino acid at a position corresponding to position 283 is substituted with Ala, Arg, Asn, Asp, Cys, Gln, Glu, Gly, His, Ile, Leu, Lys, Met, Phe, Pro, Ser, Thr, Trp, Tyr, or Val, preferably with Gly In another aspect, the variant comprises or consists of the substitution S283G of the mature polypeptide of SEQ ID NO: 6.

In another aspect, the variant comprises or consists of a substitution at a position corresponding to position 456. In another aspect, the amino acid at a position corresponding to position 456 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu. In another aspect, the variant comprises or consists of the substitution N456E of the mature polypeptide of SEQ ID NO: 6.

In another aspect, the variant comprises or consists of a substitution at a position corresponding to position 512. In another aspect, the amino acid at a position corresponding to position 512 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another aspect, the variant comprises or consists of the substitution F512Y of the mature polypeptide of SEQ ID NO: 6.

In another aspect, the variant comprises or consists of a substitution at positions corresponding to positions 100 and 283, such as those described above.

In another aspect, the variant comprises or consists of substitutions at positions corresponding to positions 100 and 456, such as those described above.

In another aspect, the variant comprises or consists of substitutions at positions corresponding to positions 100 and 512, such as those described above.

In another aspect, the variant comprises or consists of substitutions at positions corresponding to positions 283 and 456, such as those described above.

In another aspect, the variant comprises or consists of substitutions at positions corresponding to positions 283 and 512, such as those described above.

In another aspect, the variant comprises or consists of substitutions at positions corresponding to positions 456 and 512, such as those described above.

In another aspect, the variant comprises or consists of substitutions at positions corresponding to positions 100, 283, and 456, such as those described above.

In another aspect, the variant comprises or consists of substitutions at positions corresponding to positions 100, 283, and 512, such as those described above.

In another aspect, the variant comprises or consists of substitutions at positions corresponding to positions 100, 456, and 512, such as those described above.

In another aspect, the variant comprises or consists of substitutions at positions corresponding to positions 283, 456, and 512, such as those described above.

In another aspect, the variant comprises or consists of substitutions at positions corresponding to positions 100, 283, 456, and 512, such as those described above.

In another aspect, the variant comprises or consists of one or more (several) substitutions selected from the group consisting of G142S, Q183R, H266Q, and D703G.

In another aspect, the variant comprises or consists of the substitutions F100D+S283G of the mature polypeptide of SEQ ID NO: 6.

In another aspect, the variant comprises or consists of the substitutions F100D+N456E of the mature polypeptide of SEQ ID NO: 6.

In another aspect, the variant comprises or consists of the substitutions F100D+F512Y of the mature polypeptide of SEQ ID NO: 6.

In another aspect, the variant comprises or consists of the substitutions S283G+N456E of the mature polypeptide of SEQ ID NO: 6.

In another aspect, the variant comprises or consists of the substitutions S283G+F512Y of the mature polypeptide of SEQ ID NO: 6.

In another aspect, the variant comprises or consists of the substitutions N456E+F512Y of the mature polypeptide of SEQ ID NO: 6.

In another aspect, the variant comprises or consists of the substitutions F100D+S283G+N456E of the mature polypeptide of SEQ ID NO: 6.

In another aspect, the variant comprises or consists of the substitutions F100D+S283G+F512Y of the mature polypeptide of SEQ ID NO: 6.

In another aspect, the variant comprises or consists of the substitutions F100D+N456E+F512Y of the mature polypeptide of SEQ ID NO: 6.

In another aspect, the variant comprises or consists of the substitutions S283G+N456E+F512Y of the mature polypeptide of SEQ ID NO: 6.

In another aspect, the variant comprises or consists of the substitutions F100D+S283G+N456E+F512Y of the mature polypeptide of SEQ ID NO: 6.

The variants may consist of 720 to 863 amino acids, e.g., 720 to 739, 740 to 759, 760 to 779, 780 to 799, 800 to 819, 820 to 839, and 840 to 863 amino acids.

In one aspect, a variant beta-glucosidase comprises or consists of the mature polypeptide of SEQ ID NO: 36.

The variants may further comprise an alteration at one or more (several) other positions.

In one embodiment, the amount of cellobiohydrolase I in an enzyme composition of the present invention is 5% to 60% of the total protein of the enzyme composition, e.g., 7.5% to 55%, 10% to 50%, 12.5% to 45%, 15% to 40%, 17.5% to 35%, and 20% to 30% of the total protein of the enzyme composition.

In another embodiment, the amount of cellobiohydrolase II in an enzyme composition of the present invention is 2.0-40% of the total protein of the enzyme composition, e.g., 3.0% to 35%, 4.0% to 30%, 5% to 25%, 6% to 20%, 7% to 15%, and 7.5% to 12% of the total protein of the enzyme composition.

In another embodiment, the amount of beta-glucosidase in an enzyme composition of the present invention is 0% to 30% of the total protein of the enzyme composition, e.g., 1% to 27.5%, 1.5% to 25%, 2% to 22.5%, 3% to 20%, 4% to 19%, % 4.5 to 18%, 5% to 17%, and 6% to 16% of the total protein of the enzyme composition.

In another embodiment, the amount of AA9 polypeptide in an enzyme composition of the present invention is 0% to 50% of the total protein of the enzyme composition, e.g., 2.5% to 45%, 5% to 40%, 7.5% to 35%, 10% to 30%, 12.5% to 25%, and 15% to 25% of the total protein of the enzyme composition.

In another embodiment, the amount of xylanase in an enzyme composition of the present invention is 0% to 30% of the total protein of the enzyme composition, e.g., 0.5% to 30%, 1.0% to 27.5%, 1.5% to 25%, 2% to 22.5%, 2.5% to 20%, 3% to 19%, 3.5% to 18%, and 4% to 17% of the total protein of the enzyme composition.

In another embodiment, the amount of beta-xylosidase in an enzyme composition of the present invention is 0% to 50% of the total protein of the enzyme composition, e.g., 0.5% to 30%, 1.0% to 27.5%, 1.5% to 25%, 2% to 22.5%, 2.5% to 20%, 3% to 19%, 3.5% to 18%, and 4% to 17% of the total protein of the enzyme composition.

In another embodiment, the amount of endoglucanase I in an enzyme composition of the present invention is 0.5% to 30% of the total protein of the enzyme composition, e.g., 1.0% to 25%, 2% to 20%, 4% to 25%, 5% to 20%, 16% to 15%, and 7% to 12% of the total protein of the enzyme composition.

In another embodiment, the amount of endoglucanase II in an enzyme composition of the present invention is 0.5% to 30% of the total protein of the enzyme composition, e.g., 1.0% to 25%, 2% to 20%, 4% to 25%, 5% to 20%, 16% to 15%, and 7% to 12% of the total protein of the enzyme composition.

### Enzymatic Amount

In particular embodiments, the protease is present in the enzyme composition in a range of about 10% w/w to about 65% w/w of the total amount of enzyme protein. In other embodiments, the protease is present in about 10% w/w to about 60% w/w, about 10% w/w to about 55% w/w, about 10% w/w to about 50% w/w, about 15% w/w to about 65% w/w, about 15% w/w to about 60% w/w, about 15% w/w to about 55% w/w, about 15% w/w to about 50% w/w, about 20% w/w to about 65% w/w, about 20% w/w to about 60% w/w, about 20% w/w to about 55% w/w, about 20% w/w to about 50% w/w, about 25% w/w to about 65% w/w, about 25% w/w to about 60% w/w, about 25% w/w to about 55% w/w, about 25% w/w to about 50% w/w, about 30% w/w to about 65% w/w, about 30% w/w to about 60% w/w, about 30% w/w to about 55% w/w, about 30% w/w to about 50% w/w, about 35% w/w to about 65% w/w, about 35% w/w to about 60% w/w, about 35% w/w to about 55% w/w, or about 35% w/w to about 50% w/w.

Enzymes may be added in an effective amount, which can be adjusted according to the practitioner and particular process needs. In general, enzyme may be present in an amount of 0.0001-1 mg enzyme protein per g dry solids (DS) kernels, such as 0.001-0.1 mg enzyme protein per g DS kernels. In particular embodiments, the enzyme may be present in an amount of, e.g., 1 μg, 2.5 μg, 5 μg, 10 μg, 20 μg, 25 μg, 50 μg, 75 μg, 100 μg, 125 μg, 150 μg, 175 μg, 200 μg, 225 μg, 250 μg, 275 μg, 300 μg, 325 μg, 350 μg, 375 μg, 400 μg, 450 μg, 500 μg, 550 μg, 600 μg, 650 μg, 700 μg, 750 μg, 800 μg, 850 μg, 900 μg, 950 μg, 1000 μg enzyme protein per g DS kernels.

### Other Enzyme Activities

According to the invention an effective amount of one or more of the following activities may also be present or added during treatment of the kernels: catalase, pentosanase, pectinase, arabinanase, arabinofurasidase, xyloglucanase, phytase activity.

It is believed that after the division of the kernels into finer particles the enzyme(s) can act more directly and thus more efficiently on cell wall and protein matrix of the kernels. Thereby the starch is washed out more easily in the subsequent steps.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure, including definitions will be controlling.

Various references are cited herein, the disclosures of which are incorporated by reference in their entireties.

### Examples

### Materials and Methods

### Enzymes:

Protease I: Acidic protease from Aspergillus aculeatus, CBS 101.43 disclosed in WO 95/02044.
Protease A: Aspergillus oryzae aspergillopepsin A, disclosed in Gene, vol. 125, issue 2, pages 195-198 (30 Mar. 1993).
Protease B: A metalloprotease from Thermoascus aurantiacus (AP025) having the mature acid sequence shown as amino acids 1-177 SEQ ID NO: 2 in WO2003/048353-A1.
Protease C: Rhizomucor miehei derived aspartic endopeptidase produced in Aspergillus oryzae (Novoren™) available from Novozymes A/S, Denmark.
Protease D: S53 protease 3 from Meripilus giganteus disclosed in WO 2014/037438 (SEQ ID NO: 6).
Cellulase J: A blend of a Trichophaea saccata GH10 xylanase (WO 2011/057083) and Talaromyces emersonii beta-xylosidase with a Trichoderma reesei cellulase preparation containing Aspergillus fumigatus cellobiohydrolase I (WO 2011/057140), Aspergillus fumigatus cellobiohydrolase II (WO 2011/057140), Aspergillus fumigatus beta-glucosidase variant (WO 2012/044915), and Penicillium sp. (emersonii) GH61 polypeptide (WO 2011/041397).
Cellulase K: A Trichoderma reesei cellulase preparation containing Trichophaea saccata GH10 xylanase (WO 2011/057083) and Talaromyces emersonii beta-xylosidase.

### Additional/Comparative Cellulases

The below enzymes are disclosed in e.g., WO 2014/082566.

Cellulase A: A blend of an Aspergillus aculeatus GH10 xylanase (WO 94/021785) and a Trichoderma reesei cellulase preparation containing Aspergillus fumigatus beta-glucosidase (WO 2005/047499) and Thermoascus aurantiacus GH61A polypeptide (WO 2005/074656).

Cellulase B: A Trichoderma reesei cellulase preparation containing Aspergillus oryzae betaglucosidase fusion protein (WO 2008/057637) and Thermoascus aurantiacus GH61A polypeptide (WO 2005/074656).

Cellulase C: A blend of an Aspergillus fumigatus GH10 xylanase (WO 2006/078256) and Aspergillus fumigatus beta-xylosidase (WO 2011/057140) with a Trichoderma reesei cellulase preparation containing Aspergillus fumigatus cellobiohydrolase I (WO 2011/057140), Aspergillus fumigatus cellobiohydrolase II (WO 2011/057140), Aspergillus fumigatus beta-glucosidase variant (WO 2012/044915), and Penicillium sp. (emersonh) GH61 polypeptide (WO 2011/041397).

Cellulase D: Aspergillus aculeatus GH10 xylanase (WO 94/021785).

Cellulase E: A Trichoderma reesei cellulase preparation containing Aspergillus aculeatus GH10 xylanase (WO 94/021785).

Cellulase F: A Trichoderma reesei cellulase preparation containing Aspergillus fumigatus GH10 xylanase (WO 2006/078256) and Aspergillus fumigatus beta-xylosidase (WO 2011/057140).

Cellulase G: A cellulolytic enzyme composition containing Aspergillus aculeatus Family 10 xylanase (WO 1994/021785) and cellulolytic enzyme composition derived from Trichoderma reesei RutC30.

Cellulase H: A cellulolytic composition derived from Trichoderma reesei RutC30.

### Methods

### Determination of Protease HUT Activity:

1 HUT is the amount of enzyme which, at 40° C. and pH 4.7 over 30 minutes forms a hydrolysate from digesting denatured hemoglobin equivalent in absorbancy at 275 nm to a solution of 1.10 μg/ml tyrosine in 0.006 N HCl which absorbancy is 0.0084. The denatured hemoglobin substrate is digested by the enzyme in a 0.5 M acetate buffer at the given conditions. Undigested hemoglobin is precipitated with trichloroacetic acid and the absorbance at 275 nm is measured of the hydrolysate in the supernatant.

### Example 1. Screening Assay

High-throughput screening is used to evaluate enzymes for starch releasing activity. Purified enzymes are screened for their ability to improve starch release from knife milled corn fiber. Xylanases and/or beta-xylosidases are tested in a background of enzymes for 18 hours at 52° C. and pH 4.

Step 1: Incubate for 16 hours at 52° C. and pH 4. Add 200 uL substrate (3.5% solids) to filter plate (100 um nylon mesh plate) placed over receiver plate containing 5 mm glass bead. Add 100 uL water over top of substrate. Allow to strain through.

Step 2: Wash solids and combine filtrates. Wash solids (8×200 uL water) by gravity with mixing and a final spin at 1000 rpm for 1 minute. Combine 200 uL from receiver plate with 1600 uL from washings.

Step 3: Isolate starch. Pellet starch by centrifugation (3000 rpm for 3 minutes). Remove 1600 uL supernatant by automated aspiration.

Step 4: Treat with alpha-amylase/glucoamylase and measure glucose. Measure background glucose. Re-suspend starch pellet and incubate with alpha-amylase (95° C., 6 minute) then glucoamylase (50° C., 30 minutes). Measure glucose, and subtract background measurement.

| | Starch release | Starch release |
|---|---|---|
| | (2 mg/g DS) | (0.5 mg/g DS) |
| Aspergillus fumigatus GH10 | 0.26 | 0.12 |
| xylanase | | |
| Trichophaea saccata GH10 | 0.41 | 0.20 |
| xylanase (trial 1) | | |
| Trichophaea saccata GH10 | 0.37 | 0.33 |
| xylanase (trial 2) | | |

T. sacchata GH10 xylanase shows improvement compared to A. fumigatus GH10 xylanase over a background blend of CELLUCLAST®/Protease B.

### Example 2. Assay to Release Starch from Corn Fiber

1-g fiber (knife-milled) assay, using 2 mg/g (total protein) or 300 ug/g (purified xylanase protein), incubation for 2 hrs at 52° C. and pH 4. Procedure:

1. Measure dry solids of washed fiber.
2. Weigh into each 50-ml centrifuge tube 1.0 g (dry solids) of fiber and record actual weight.
3. Add deionized water and buffer to make up a final volume of 25 ml.
4. Prepare enzymes according to dilution.
5. Dose enzymes into tubes.
6. Incubate tubes in rotisserie ovens for four hours, 52° C.
7. Remove tubes from oven and allow to cool to room temperature.
8. Filter the slurry through the filter unit. Transfer as much fiber as practical into the funnel with help of a spatula.
9. Aid dewatering by gently mixing and pressing with spatula.
10. Unscrew the filter centrifuge tube and transfer the filter unit to the empty incubation tube.
11. Cap the filter tube and centrifuge at 2500 rpm for 5 min.
12. Decant the supernatant without disturbing the pellets.
13. Replace the filtration unit back to the filter tube.
14. Slowly add 30 ml deionized water into each funnel while stirring with the spatula.
15. Repeat steps 9 to 13.
16. Repeat steps 14 and 15.
17. Draw as much supernatant from the pellet using a transfer pipette.
18. Transfer the retained fiber to preweighed aluminium pans.
19. Dry retained fiber overnight in 105° C. oven.
20. Dry filtrate pellet overnight in 50-55° C. oven.

### Following Day:

21. Weigh dry fiber in pan (to obtain residual dry solids)
22. Measure starch in dry filtrate using enzymatic starch assay, wherein sample is treated with a conventional alpha-amylase and glucoamylase to quantitatively convert the starch to glucose. The final glucose amount is then determined by HPLC, and then converted back to starch content value. In practice, dry solid samples less than or about 1-gram mass is suspended in buffer containing an excess of alpha-amylase, and then incubated for 2 hr at 85° C. After incubation, the samples are transferred to a 50° C. bath, and then added with an excess of glucoamylase. Additional 1 hr incubation is sufficient to convert all starch dextrins to glucose. Typical starch content of the 1-g fiber assay samples determined by this enzymatic method range from 50 to 70% of dry solids.

### Example 3. Wet Milling in the Presence of Cellulase K

The 10-g fiber assay generally includes incubating wet fiber samples obtained from wet-milling plant, in the presence of enzymes, at conditions relevant to the process (pH 3.5 to 4, Temp around 52° C.) and over a time period of between 1 to 4 hr. After incubation the fiber is transferred and pressed over a screen (typically 100 micron or smaller), where the filtrates consisting mainly of the separated starch and gluten are then collected. A number of washes are done over the screen, and the washings are collected together with the initial filtrate. The collected filtrate are then passed over a funnel filter (glass filter with 0.45 micron opening) to further separate the insoluble solids (starch and gluten) from the rest of the filtrates (mostly dissolved solids). These retained insoluble solids are washed and then oven dried to dryness. The insoluble dry mass is weighed and then analyzed for starch content.

10-g fiber assay is performed at pH 3.8, incubating at 52° C. for 1 hour at dose of 30 ug EP/g corn. Blend ratio of Cellulase F:CELLUCLAST® (available from Novozymes A/S) or Cellulase K:CELLUCLAST® is 1:1 and protease component (Protease D) is 10%.

TABLE 3
Release of starch + gluten (dry substance) from corn fiber at dose
of 30 ug/g corn.

| Treatments | Starch + Gluten Recovered |
|---|---|
| No enzyme | 8.50% |
| Cellulase F + Celluclast + Protease D | 10.15% |
| Cellulase K + Celluclast + Protease D | 11.25% |

As shown in Table 3, baseline blend of Cellulase K+Celluclast+Protease D has the best performance with 0.28% increase of starch+gluten releasing from fiber.

### Example 4. Wet Milling in the Presence of Cellulase K and Protease B

The 10-g fiber assay generally includes incubating wet fiber samples obtained from wet-milling plant, in the presence of enzymes, at conditions relevant to the process (pH 3.5 to 4, Temp around 52° C.) and over a time period of between 1 to 4 hr. After incubation the fiber is transferred and pressed over a screen (typically 100 micron or smaller), where the filtrates consisting mainly of the separated starch and gluten are then collected. A number of washes are done over the screen, and the washings are collected together with the initial filtrate. The collected filtrate are then passed over a funnel filter (glass filter with 0.45 micron opening) to further separate the insoluble solids (starch and gluten) from the rest of the filtrates (mostly dissolved solids). These retained insoluble solids are washed and then oven dried to dryness. The insoluble dry mass is weighed and then analyzed for starch content.

10-g fiber assay was performed at pH 3.8, incubating at 52° C. for 1 hour at dose of 30 ug EP/g corn. Blend ratio of Cellulase F:CELLUCLAST® (available from Novozymes A/S) or Cellulase K:CELLUCLAST® is 1:1 and protease component (Protease B) is 10%. Release of starch+gluten (dry substance) from corn fiber at dose of 30 ug/g corn was measured.

More particularly according to an exemplary 10-g fiber assay, the below equipment and reagents are used to analyze pressed corn fiber sample (sourced from wet-milling plant), which is stored frozen and thawed prior to use, according to the steps in the table:
150-μm Opening Sieves and Catch pan (Retsch GmbH)
250 ml Erlenmeyer Flask with caps
150 ml Bottles
Glass Micro filter Paper (Whatman 150 mm-Diameter)
Vacuum Filtration apparatus
Small aluminum pans
2000 ml plastic beaker
600 ml glass beaker
Funnel
Moisture analyzer
Glass vials and caps for HPLC system
HPLC system
0.45 μm pore size polypropylene syringe filters (Whatman)
3 ml plastic syringes
Oven (Capable to heat to 105° C.)
Ice bath
Analytical balance
Rubber Spatula
0.4M HCl
1M Sodium Acetate buffer (pH 4)
1M Acetic Acid
1M pH 7 Sodium Acetate

| Step | Action |
|---|---|
| 1 | Determine moisture of ~1 g corn fiber using the Moisture balance |
| | Collect the DS % |
| 2 | Weigh out items and record initial weights of |
| | Flasks, Bottles, Small Aluminum pans, Glass Micro Filter paper |
| 3 | Determine the amount of fiber that needs to be weighed out for each replicate to obtain a dry |
| | solids of 5 grams |
| 4 | After adding the fiber into the flask, store them into the cold room until ready for use |
| | Fiber can last about 2-3 days in the cold room |
| 6 | Add 98 ml of water to each flask of fiber to achieve desired % DS |
| 7 | Add 2 ml of buffer (1M pH 4.0 Sodium Acetate) to adjust pH to 4.0 (the final buffer concentration |
| | is 0.02M) |
| 8 | Add enzyme into the flask |
| 9 | Place flask into Incubator(New Brunswick Scientific/Innova 42) and set at 150RPM @50° C. |
| | for 4 hours |
| 10 | After the incubation place the flask into ice bath to slow enzyme activity |
| | Let flask sit in the ice bath for a minimum of 5 minutes |
| 11 | For each sample flask, pour out the content onto the 150 μm sieve with catch pan below |
| 12 | Measure about ~200 ml of tap water into a beaker and pour into the flask to rinse any remaining |
| | fiber, then pour the rinse water back into the beaker |
| 13 | Using the spatula, press the fiber against the screen to release water and insolubles into the |
| | catching pan. |
| 14 | Once a majority of the water has been pressed out, place the fiber back in the beaker |
| | containing the 200 ml of rinse water in Step 12 |
| 15 | Stir the fiber in the beaker with the spatula, then pour onto the 150 μm sieve |
| | Considered 1^{st} Wash |
| 16 | Measure out ~200 ml of water into the rinse beaker |
| 17 | Press the fiber again with spatula until majority of water has been pressed out, then dump |
| | fiber back into the rinse beaker |
| 18 | Remove the sieve pan and pour the liquid from the catching pan into 1 Liter Plastic Bottle |
| | Give a gentle swirl to the pan before pouring to get the sediments to go into the bottle |
| 19 | Repeat Steps 15 to 18 two more times (for a total of 3 wash steps) |
| | At the end of the 3^{rd} wash, the fiber may be discarded unless saved for additional analysis. |
| 20 | Take the 1 L bottles containing the sieve-throughs to the Manifold Vacuum Filtration setup |
| 25 | After rinsing the filter funnels with tap water, place the preweighed glass filter paper into the |
| | funnel and spray DI water to keep filter in place |
| 27 | Turn on the vacuum, then pour the entire bottle content gradually into the funnel |
| 28 | As the samples are filtering, fill the emptied bottle with ~200 ml of DI water and pour into the |
| | filter with the rest of sample |
| | Turn the Vacuum off once the solution is filter through then add the DI water to the funnel |
| | and turn the Vacuum back on |
| 29 | Once the solution is finish before the filter dry out |
| | Turn off the vacuum and pour the water into the funnel and turn the vacuum back on |
| 30 | This is removing the remaining solvents in the bottle and also rinsing the filter keeping the |
| | insolubles |
| 32 | To remove the filters use a metal spatula to lift the edge of filters up and to scrape any remaining |
| | insolubles off the sides. |
| 33 | Take the filter and fold twice and place them into the pre-weighed pan |
| 34 | Remember to weigh the pan now with the Filter paper |
| 35 | Place the pan into the 105° C. oven overnight to dry |
| 36 | Weigh out the pan with the dry filtered matter. |
| | This weight is used to calculate insoluble solids yield. |
| 37 | Remove the filter from the pan taking care that no filtered solids are lost, then cut each into |
| | strips and further into small squares to go into the glass bottle |
| | Make sure that you cut the filters into smaller pieces so that they can be remove once finish |
| 38 | Measure out 50 ml of 0.4M of HCL into each bottle |
| | Let the filter paper sit in the solution for at least 2 hours; No more than 24 hours |
| 39 | Place into the autoclave for Residual Starch procedure |
| | Autoclave needs to be set @230° F. for 80 minutes |
| 40 | Once autoclave is done let the bottle cool down before touching |
| 41 | Filter the solution into HPLC vials and send them off to be analyzed for glucose. |
| | NOTE: The glucose concentrations are used to calculate the amount of starch in the insoluble |
| | solids |

### Results:

| Blend | Starch + gluten (% DS fiber) |
|---|---|
| Control | 14.51% |
| Cellulase F + Celluclast + Protease B | 15.20% |
| Cellulase K + Celluclast + Protease B | 17.90% |

### Example 5

Cellulase L: A Trichoderma reesei Cellulase Preparation Containing a CBHI of SEQ ID NO: 2, a CBHII of SEQ ID NO: 4, a GH10 of SEQ ID NO: 10, and a Beta-Xylosidase of SEQ ID NO: 14.

The 10-g fiber assay generally includes incubating wet fiber samples obtained from wet-milling plant, in the presence of enzymes, at conditions relevant to the process (pH 3.5 to 4, Temp around 52° C.) and over a time period of between 1 to 4 hr. After incubation the fiber is transferred and pressed over a screen (typically 100 micron or smaller), where the filtrates consisting mainly of the separated starch and gluten are then collected. A number of washes are done over the screen, and the washings are collected together with the initial filtrate. The collected filtrate are then passed over a funnel filter (glass filter with 0.45 micron opening) to further separate the insoluble solids (starch and gluten) from the rest of the filtrates (mostly dissolved solids). These retained insoluble solids are washed and then oven dried to dryness. The insoluble dry mass is weighed and then analyzed for starch content.

10-g fiber assay was performed at pH 4.0, incubating at 52° C. for 1 hour at dose of 50 ug EP/g corn or 100 ug EP/g corn, using a blend of Cellulase L or Cellulase F: CELLUCLAST® (available from Novozymes A/S) in combination with Protease D.

Blend ratio of Cellulase F:CELLUCLAST® is 4:1 and protease component (Protease D) is 10%. Release of starch+gluten (dry substance) from corn fiber at the specified doses below was measured.

### Results:

| | Dose (ug enzyme | Starch + |
|---|---|---|
| Treatments | protein/g corn) | Gluten Recovered |
| No enzyme | 0 | 6.36% |
| Cellulase F + Celluclast + | 50 | 8.49% |
| Protease D | | |
| Cellulase L + Protease D | 50 | 9.35% |
| Cellulase L + Protease D | 100 | 10.24% |

1-16. (canceled)

17. A process for treating crop kernels, comprising the steps of:
a) soaking kernels in water to produce soaked kernels;
b) grinding the soaked kernels;
c) treating the soaked kernels in the presence of an effective amount of an enzyme composition comprising a protease, and a cellulolytic composition comprising: (A) (i) a cellobiohydrolase I, (ii) a cellobiohydrolase II, and (iii) at least one enzyme selected from the group consisting of a beta-glucosidase or a variant thereof, an AA9 polypeptide having cellulolytic enhancing activity, a GH10 xylanase, and a beta-xylosidase; (B) (i) a GH10 xylanase and (ii) a beta-xylosidase; or (C) (i) a cellobiohydrolase I, (ii) a cellobiohydrolase II, (iii) a GH10 xylanase, and (iv) a beta-xylosidase; wherein
the cellobiohydrolase I has at least 70% sequence identity to the mature polypeptide of SEQ ID NO: 2;
the cellobiohydrolase II has at least 70% sequence identity to the mature polypeptide of SEQ ID NO: 4;
the beta-glucosidase has at least 70% sequence identity to the mature polypeptide of SEQ ID NO: 6;
the xylanase has at least 70% sequence identity to the mature polypeptide of SEQ ID NO: 10 or the mature polypeptide of SEQ ID NO: 12; and
the beta-xylosidase has at least 70% sequence identity to the mature polypeptide of SEQ ID NO: 14; and
wherein step c) is performed before, during or after step b).

18. The process of claim 17, wherein the protease is present in a range of about 10% w/w to about 65% w/w of the total amount of enzyme protein.

19. The process of claim 17, wherein the protease is present in less than about 60% w/w of the enzyme composition.

20. The process of claim 17, wherein the protease is present in about 50% w/w of the total amount of enzyme protein.

21. The process of claim 17, wherein the protease is present in about 25% w/w of the total amount of enzyme protein.

22. The process of claim 17, wherein the kernels are soaked in water for about 2-10 hours.

23. The process of claim 17, wherein the soaking is carried out at a temperature between about 40° C. and about 60° C.

24. The process of claim 17, wherein the soaking is carried out at acidic pH.

25. The process of claim 17, wherein the soaking is performed in the presence of between 0.01-1% SO₂ and/or NaHSO₃.

26. The process of claim 17, wherein the crop kernels are from corn (maize), rice, barley, sorghum bean, or fruit hulls, or wheat.
